(19) United States
(12) Patent Application Publication
(10) Pub. No.: US 2022/0392596 A1
(43) Pub. Date: Dec. 8, 2022

(54) System and Method for Treating Migraine and Headache Through a Digital Therapeutic

(71) Applicant: Head Health, Inc., Wheaton, IL (US)

(72) Inventors: Anthony Cocco, Streamwood, IL (US); Bonita Cocco, Wheaton, IL (US); Amit Patel, Bucyrus, KS (US); Luyaun Fang, Seattle, WA (US); Fabio Chiussi, Seattle, WA (US); Andrew Sampson, Channahon, IL (US)

(21) Appl. No.: 17/834,396

(22) Filed: Jun. 7, 2022

Related U.S. Application Data
(60) Provisional application No. 63/197,689, filed on Jun. 7, 2021.

(51) Int. Cl. G16H 20/00; A61B 5/00; G16H 10/60; G16H 50/20; G16H 50/30; G16H 80/00

(52) U.S. Cl. CPC G16H 20/00; A61B 5/7275; G16H 10/60; G16H 50/20; G16H 50/30; G16H 80/00

(57) ABSTRACT

A system and method for treating migraine and headache through a digital therapeutic. The systems and methods described herein allow a migraine patient to manage symptoms, triggers, and therapeutic lifestyle interventions through an interconnected system, facilitated by a care team, to treat the root causes of migraine, and improve patient quality of life. The method includes collecting datasets associated with migraine, headache, migraine-related comorbidities, and lifestyle behavior at the mobile device to analyze neurological trigger patterns. Using the digital therapeutic application allows a patient to monitor and track lifestyle habits, to receive real time interventions, otherwise missed in traditional care settings, helping the patient learn lifestyle interventions that may mitigate future attacks. Given the complex nature of treating migraine, therapeutic lifestyle interventions are tailored to a patient's Neuroindividuality and facilitated by a care team.

### TECHNICAL FIELD

The present invention relates generally to the digital health field, but more specifically to new methods and systems of treating migraine and headache through a digital therapeutic.

### BACKGROUND

Migraine is a severe brain condition, categorized as the sixth most disabling global disorder by the World Health Organization. Neurological disorders ranked as the leading cause of disability-adjusted life-years with the most prevalent disorders being tension-type headache, migraine, medication overuse headache. Migraine predominantly affects females and negatively impacts the quality of life.

Characterized by throbbing head pain, sensitivity to visual and sound, and accompanied by additional symptoms such as irritability, reduced concentration, and tiredness. Patients commonly report foods, alcohol, meteorologic or atmospheric changes, exposure to light, sounds, or odors, as factors that trigger or aggravate their migraine attacks. Migraineurs tend to have high levels of depression and anxiety.

Studies have supported the association of migraine and comorbid mental health conditions. Migraineurs have an increase in lifetime rates of anxiety disorders and depression proving to be another opportunity for misdiagnosis. As the stigma and isolation become more consistent, migraineurs often turn to social media for connection which unfortunately may exacerbate symptoms or contribute to anxiety-related dizziness.

The diversity of symptoms experienced by migraine patients requires a complex neurological evaluation, involving multiple neurochemical, neuropsychological, chemical, hormonal, and neurobiological processes. Migraine is a multifaceted neurobiological phenomenon that involves the activation of diverse neurochemical and cellular signaling pathways in multiple regions of the brain.

Migraine imposes high direct and indirect burden due to significantly higher costs than controls. Migraineurs have 2.5 times as many pharmaceutical claims, twice as many medical claims, and more emergency room services. Patients have significantly greater disease comorbidity and higher use of prescription medications and controlled painkillers at rates that raise the concern of overuse.

An unmet need exists for new treatment modalities and may lead to improved patient outcomes and lower healthcare costs.

Given the difficulties with diagnosis and pharmacological efficacy for migraine, migraineurs are an ideal population to study alternative practices and prevention.

Extensive preclinical studies have identified neurochemical, cellular, hormonal, neuropsychological, and neurobiological targets for potential intervention. Identification of environmental factors may be helpful to reduce attack frequency. There is a strong need in clinical practice for alternative approaches for both acute and preventive treatment, including digital therapeutic.

Embodiments of the present invention include a comprehensive personalized digital therapeutic based on the underlying pathogenesis of migraine and involves multiple modalities designed to achieve an improved quality of life. Embodiments of the present invention are designed to fill the unmet need for a functional wellness treatment approach to migraine, headache, combination headache types, and to the limitations of the current clinical approach.

### SUMMARY

Migraine is one of the most complex neurological disorders. Our technology and systems are designed to recognize the diversity of symptoms, triggers, headache types experienced by migraine patients. With treatment systems available today, many healthcare professionals do not have the necessary time to perform a full analysis of the whole-body care required to improve the quality of life for a migraine and headache patient. If a migraine or headache sufferer is non-responsive to current treatments and/or diagnostic tests are negative, our technology will help identify trends in lifestyle and/or behavior to provide insights to possible triggers creating the symptom and/or pain. If a user experiences health conditions such as allergy, sinus, anxiety, and/or depression, lifestyle monitoring can recognize trends in symptoms, pain, frequency, and/or duration to make personalized recommendations to improve access to care and lifestyle interventions.

With the digital therapeutic we will address accessibility and real-time intervention and diagnose patients as a whole and with the personal attention and information that the patient needs. Embodiments of our invention will identify trends in the patterns of migraine and headache sufferers to provide behavioral health and lifestyle interventions using digital technology. We help a patient identify the root causes of migraine, headache, and/or underlying health conditions triggering the frequency, duration, and/or pain to create a dynamic patient experience based on their unique neurological health profile.

If a patient's migraine is triggered by stress, mental health, and blood sugar imbalances, by identifying trends in lifestyle through interacting with the invention, specific interventions and notifications will be delivered to mitigate in real-time. Actionable steps will be provided along with monitoring to determine the success of the user's intervention.

If a patient experiences physical exertion headache, the technology will cross reference health history and lifestyle patterns. Specific questions will be delivered to determine likelihood of medical intervention. Depending on answers, the user will receive dynamic recommendations for new physical activity recommendations, breathing exercises, and content to support this process.

As the patients continuing to provide both passive and active feedback, the analytics data engine will cross-reference and match probability.

### BRIEF DESCRIPTION OF THE DRAWINGS

FIG. 1: Schematic diagram of a computer system providing therapeutic interventions for migraine and headache according to an embodiment of the present invention

FIG. 2: Schematic diagram of an embodiment for the method to deliver therapeutic lifestyle intervention for migraine and headache across the system according to an embodiment of the present invention

FIG. 3: Exemplary illustration of the migraine headache data collector according to an embodiment of the present invention

FIG. 4: Exemplary illustration of the migraine headache analyzer according to an embodiment of the present invention

FIG. 5: Exemplary illustration of the intervention predictor according to an embodiment of the present invention

FIG. 6A-6E: Exemplary illustration of the lifestyle intervention manager according to an embodiment of the present invention

FIG. 7: Exemplary illustration of the intervention notification manager according to an embodiment of the present invention

FIG. 8: Schematic flow diagram of the method for the Neuroindividuality module in accordance with the present invention

FIG. 9: Schematic flow diagram of the method for the trigger analysis module in accordance with the present invention

FIG. 10: Schematic flow diagram of the method for the headache type probability analysis module in accordance with the present invention

FIG. 11: Schematic flow diagram of the method for the therapeutic comparison module in accordance with the present invention

FIG. 12: Schematic flow diagram of the method for the root cause analysis module in accordance with the present invention

FIG. 13: Schematic flow diagram of the method for the adherence analysis module in accordance with the present invention

FIG. 14: Schematic flow diagram of the method for the feedback analysis module in accordance with the present invention

FIG. 15: Schematic flow diagram of the method for the prediction analysis module in accordance with the present invention

FIG. 16: Schematic flow diagram of the method for the intervention generation module in accordance with the present invention

FIG. 17: Schematic flow diagram of the method for the notification manager module in accordance with the present invention

FIG. 18: Schematic flow diagram of the method for the recommendation module in accordance with the present invention

FIG. 19: Schematic flow diagram of the method for the notification scheduler in accordance with the present invention

FIG. 20: Exemplary illustration of the application of the therapeutic intervention prompt according to an embodiment of the present invention

FIG. 21: System view of the process to generate feedback according to an embodiment of the present invention

### DETAILED DESCRIPTION

The present invention comprises systems and methods for treating migraine and headache through a digital therapeutic and will perform at least the following:
Collecting log of use, lifestyle, food intake, and symptom datasets associated with user migraine and headache behavior at the mobile device to analyze migraine, headache, and neurological trigger patterns
Receiving user data to execute a basic model of the neurological system to generate a modified therapeutic intervention
Retrieving a migraine headache trigger risk model
Retrieving a Neuroindividuality predictability model
Performing a statistical analysis to predict a readiness factor score and symptom threshold in response to communication patterns, known triggers, and change in behavior
Determining a migraine and headache status and probability analysis of the user based on one or more of the datasets
Predicting a risk of change in the trigger, symptom, communication, and lifestyle behavior for users
Using the model to develop a therapeutic treatment recommendation to mitigate symptoms, pain, and nervous system activity in correlation to migraine and headache
Generating readiness factor to determine user's likelihood of lifestyle adherence
Notifying interventions based on user's lifestyle activity and migraine headache patterns
Alert system to provide real-time migraine and headache therapeutic interventions in accordance with a user's connected calendar, migraine health profile, readiness factor, stress score, and log of use
Personalized therapeutic recommendation according to symptoms, triggers, lifestyle patterns, and migraine headache profile
Medication side effect and unintended consequences alerts about a user's symptoms
Monitor trends in sleep behavior and routines as it relates to the trigger of migraine and/or headache symptoms
Access to select health coach based on neurological health profile
Transmitting user data to care team
Receive user data from clinicians and healthcare professionals
Submitting migraine and headache lifestyle and user data to approved and consenting parties

### 1. System

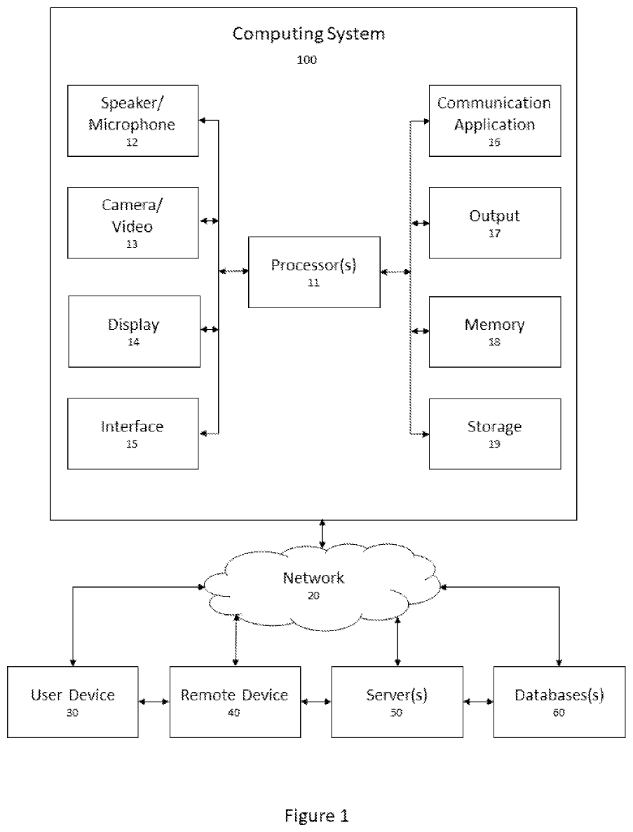

FIG. 1 shows a schematic diagram of a computer system 100 providing therapeutic interventions for migraine and headache embodying the present invention. The number of components in system 100 is provided for the purposes of illustration.

As shown in FIG. 1, in some embodiments, system 100 may include a processor 10, a speaker/microphone 11, a camera/video 12, a display 13, an interface 14, a communication application 15, an output 16, memory 17, storage 18, a network 20, a user device 30, a remote device 40, a server 50, and a database 60. The user may operate a user device 30 to communicate to and/or through the network 20 with other system components 100, such as a remote device 40 and/or the database 60, via network 20 and/or server 50. The user device 30 may include one or more devices such as a desktop computer, laptop computer, smartphone, tablet, electronic reader, wearable device, tracking device, smart watch, smart band, or other types of electronics or communication devices.

### 2. Overview of Model

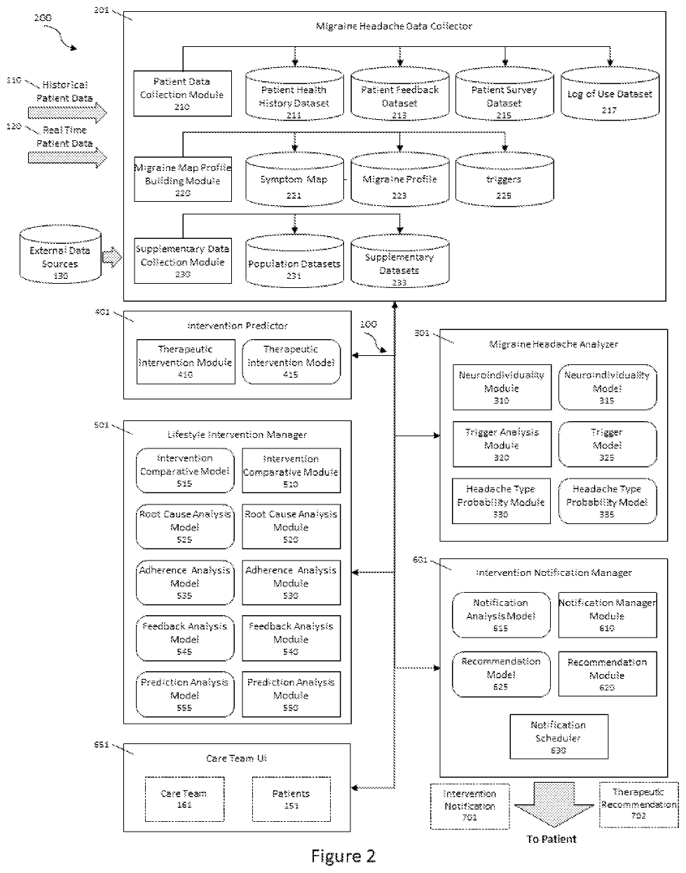

As shown in FIG. 2, the method 200 for treating migraine and headache with a digital therapeutic includes receiving historical patient data 110, real time patient data 120 and external data sources 130

Historical patient data 110 will refer to patient data that has been collected over a period of time and may include one or more of patient EHR, laboratory test results, diagnostic images, device data, medication usage, psychological data, and/or any other suitable historical patient data provided by the patient and/or collected by the system.

Real time patient data 120 will refer to data retrieved, collected, and/or processed from the system including at least one of patient mobile device, remote device, communication application, and/or care team UI 651.

External data sources 130 will refer to external data sources from servers 131, IoT devices 132, and databases 133. For example, servers 131 can include one or more of the following web servers (e.g., dynamic web pages), application servers (e.g., mobile applications), communication servers (e.g., network connectivity for communications), name servers (e.g., domain name to an IP), directory servers (e.g., network resources for security, authenticating users, groups, and devices), API servers (e.g., functionality software for IoT device or other software), database servers (e.g., access to databases), file servers (e.g., access to files), game servers (e.g., access to gamification services), and/or media servers (e.g., access to video or audio). IoT devices 132 can include one or more of the following sensors, applications, personal devices, data collectors, and any other suitable IoT device configured to integrate with the system. Databases 133 can include one or more of the following: diagnostic database (e.g., diagnostic data, list of diagnostic codes according to ICD-10, ICHD-3, diagnostic medical journals, etc.), medications database (e.g., database of medications, side effects, drug-to-drug interactions, drug interactions, allergy symptoms, etc.), genetics database (e.g., general genetic information, genome lab result data, medical journal genomic data, etc.), nutrition database (e.g., vitamins, minerals, phytonutrients, endocrine disruptors, alkaline and/or acidic characteristics, probiotic bacteria, prebiotic properties, enzymes, essential fatty acids, etc.), food and meal database (e.g., nutrition labels, ingredients, images, manufacturer information, etc.) products database (e.g., consumer products characteristics, product name, manufacturer, image, ingredients, chemicals, allergic reactions, banned substance in foreign countries, purchase options, etc.).

The method 200 is configured to process data across the migraine headache data collector 201, migraine headache analyzer 301, intervention predictor 401, lifestyle intervention manager 501, intervention notification manager 601, and the care team UI 651.

The method 200 is implemented by at least one part of the embodiment of the system 100. Alternatively, the method 200 can be used in any other suitable system configuration to process patient data to generate a model to deliver therapeutic intervention to the patient.

In another example, the method 200 is configured to receive datasets from Blocks 201, 301, 401, 501, and 601, to generate modified models to deliver digital therapeutic interventions. The method 200 can process one or more datasets to Block 651 care team UI.

The system 100 can also update the patient's application over time such as modification of models, patient datasets, patient Neuroindividuality profile, headache type probability, projected adherence analysis, therapeutic interventions, and/or predicted lifestyle regimen outcomes.

The system can implement a deep learning relationship between migraine and headache health and a patient's lifestyle in reference to improving a patient's quality of life over a length of time. The system is configured to analyze and predict a change in symptoms, pain duration, migraine comorbidity, neurological activity, and overall health. The system will assist the patient through lifestyle interventions, therapeutic interventions, targeted or automated intervention (e.g., notification, chat bot prompt, behavioral health exercise, etc.), to anticipate migraine symptoms, provide personalize care, estimate risk of intervention, and reduce hospital readmission.

In response to patient activity performed within the digital therapeutic application on the mobile computing device, the system will retrieve and/or collect data from historical patient data 110, real time patient data 120, and external data sources 130; process and send instructions to the migraine and headache data collector 201 and categorize into datasets 211, 213, 215, 217, 221, 223, 225, 231, and 233. After the data is processed and analyzed in Block 201, the system can transmit instructions to the migraine headache analyzer 301, intervention predictor 401, lifestyle intervention manager 501, intervention notification manager 601, and care team UI 651. Blocks 201, 301, 401, 501, 601, and 651 are configured by the system to deliver intervention notification 701 and therapeutic recommendation 702 to the patient. The method 200 is configured by the system to automatically and/or over a specified time period update the therapeutic delivery module in accordance with patient quality of life.

In an example, if the user is having a decrease in blood sugar, potentially triggering a blood sugar headache, the system 100 will send instructions to the method 200 to access supplement data, marketplace inventory, patient eating and cooking habits, log of use, and retrieve names of products or food items to deliver a prompt via the therapeutic delivery module (e.g., push notification, alert, etc.) to notify user of a decrease in blood sugar triggering a potential blood sugar headache with the notification to make a purchase.

Each of the exemplary modules 201, 301, 401, 501, 601, 651 in FIG. 2 is now described in turn.

### 3. Migraine Headache Data Collector

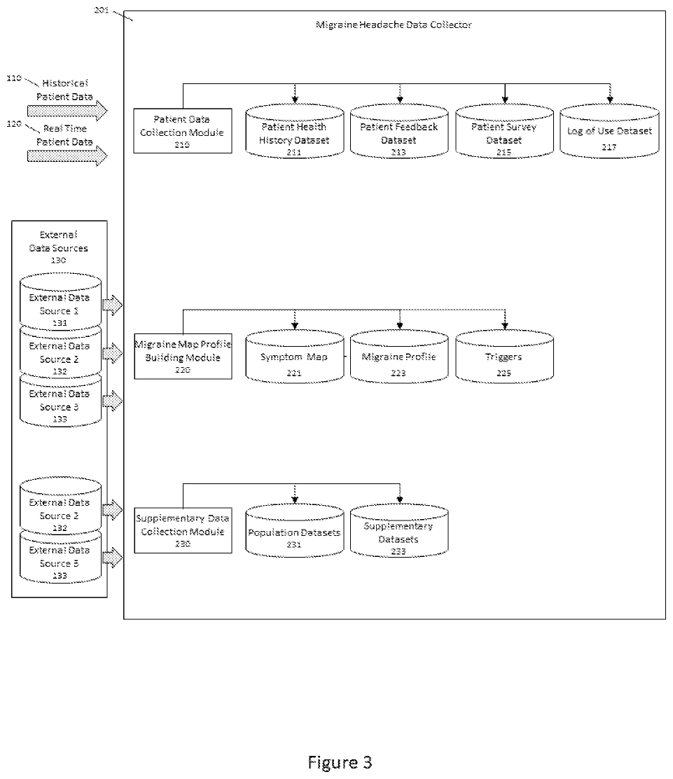

As shown in FIG. 3, Block 201 Migraine Headache Data Collector receives, collects, and/or processes Historical Patient Data 110, Real Time Patient Data 120, and External Data Sources 130 from a patient's mobile communication device. Block 201 is implemented using Patient Data Collection Module 210, Migraine Map Profile Building Module 220, and Supplementary Data Collection Module 230 configured to interface with the native digital therapeutic data collection application executing a patient's mobile communication device (e.g., smartphone, tablet, wearable computing device, vehicle, etc.) to retrieve patient data.

Block 201 can process data from Blocks 210, 220, and 230 separately and/or merged with subsequent blocks of the method 200 to deliver therapeutic interventions to a patient's mobile communicating device and/or Block 651.

In implementing Block 201, the mobile computing device can upload data to a database (e.g., cloud computing system, remove server, storage module, etc.), at a desired period of time (e.g., historical, real-time, morning, afternoon, weekend, hourly, day, month, year, etc.), and to be accessed by the system 100 and method 200.

In a variation of the example, Block 201 can take the existing data retrieved from historical patient data 110; real time patient data 120; patient health history dataset 211; log of use dataset 217; symptom map 231; migraine profile 233; triggers 235; and generate a comparison model including external data sources 130; population datasets 231; and supplementary datasets 233.

For example, the system 100 can send instructions to generate a comparison of patient health history dataset 211 and external data sources 130 (e.g., migraine and IBS medical journals, IBS treatments, IBS medications, etc.), population datasets 231 (e.g., patients who experience migraine, headache, digestive symptoms, and have chronic frequency), and supplementary datasets 233 (therapeutic intervention with IoT datasets, product purchase history, and biofeedback signals). In this example, the system 100 and method 200 can send instructions to Block 201 to prepare and transmit illustrations representing trends to the patient's mobile computing device, prompting patient survey, patient feedback, or any other suitable patient activity.

Additionally, Block 201 is configured to manage interactions with the application registered on the patient's device to facilitate the personalized communication of content and data gathering. In one example, Block 210 reported a history of anxiety, tension, and digestive discomfort; and Block 220 reported symptoms of anxiety and tension; high probability of migraine with comorbid anxiety and IBS; and seven different lifestyle triggers. With log of use dataset 217 analysis, the patient had increased adherence and activity preference to journaling and deep breathing first thing in the morning. The system 10 will communicate with the method 200, modify patient 151 instructions, and transmit data to care team 161. The system 100 can additionally update the Neuroindividuality module 310 and modify the home screen settings to suggest a journaling prompt in accordance with the notification analysis and recommendation output, as discussed in Blocks 401, 501, and 601.

The exemplary migraine headache data collector 201 shown in FIG. 2 comprises at least the following:

### 3.1 Patient Data Collection Module

The Patient Data Collection Module 210 functions as a module, to be accessed by the system 100 and processed by Block 201. Block 210 is implemented using a module and configured to interface with the digital therapeutic application executing on a patient's mobile communication device and/or care team UI 651 to retrieve at least one of an output of the historical patient data 110, real time patient data 120, and external data sources 130; and store in one or more of datasets: patient health history dataset 211, patient feedback dataset 213, patient survey dataset 215, and log of use dataset 217.

Patient health history dataset 211 is configured to receive data associated with a patient health history profile (e.g., age, height, weight, occupation, ancestry, blood type, medications, vitamins, eating habits, physical activity, and sleep). In relation to receiving the patient health history dataset 211, Block 210 can include accessing the historical patient data 110, real time patient data 120, and external data sources 130, as shown in FIG. 3. The system can be implemented in one more processing capability to transmit the health history dataset 211 in a suitable manner relating to the method 200 and instructions for Block 201.

For example, Block 210 retrieves historical patient data 110 (e.g., EHR data with height, blood type, and medications), real time patient data 120 (e.g., updated vitamin regimen and physical activity), and external data sources 130 (e.g., IoT sensor data for heart rate, weight, body fat percentage, and metabolic age); analyzes responses, and categorizes according to instructions for patient health history dataset 211.

Feedback datasets 213 can include one or more feedback responses to patient digital therapeutic application activity (e.g., positive experience with a care team member, purchasing experience, content delivery, etc.), activity interest data (e.g., nutrition, complementary and alternative medicine activities, spirituality, brain health, activities performed at home, activities with patient's children, activities at night, activities in the morning, activities during the day, indoor activities, outdoor activities, activities in a group, social-distance activities, virtual activities, etc.), goal data (e.g., general areas of concern for the user such as pain, frequency and duration or migraine headache type, triggers, nutrition, exercise, stress, social interaction, activity, anxiety, depression, sleep, menstrual migraine, premenstrual syndrome, consuming new food, emotional health, spiritual growth, weight loss or gain, focus, happiness, memory, relaxation, mindfulness, willpower, improving quality of life, attending a family function, going on a date night, relationships, care, etc.), goal target data (e.g., increase or decrease therapeutic content in relation of improving patient quality of life), lifestyle likeability data for user interest in a specific activity (e.g., journaling, meditation, exercise, cooking, eating, learning about cognitive distortions, etc.). Additionally, Block 210 is configured to analyze patient feedback data 213 and modify models in method 200 to optimize user experience.

In continuation of the previous example, Block 210 is configured to analyze patient health history dataset 211 according to data collected from historical patient data 110, real time patient data 120, and external data sources 130. Block 210 will process patient health history dataset 211 (e.g., patient has 10 years of EHR data, female, 5′5″, 160 lbs., married, two children, stay-at-home mom, Middle Eastern ancestry, blood type A−, currently on two medications with a history of eight previously used, daily vitamin regimen consisting of multivitamin and magnesium, 50% of food is home cooked with the other 50% being takeout, minimal physical activity, and poor sleep hygiene) and analyze results to generate feedback responses.

For example, Block 210 can identify one or more potential feedback responses according to the patient health history dataset 211 (e.g., interest level for activities with children, reduce medication consumption, vitamins, low impact exercises for blood type A, and nighttime sleep challenge). Patient feedback responses will be stored in the patient feedback dataset 213 and processed by Block 210. The system 100 can generate modified instructions for Block 201 to facilitate the retrieval of additional information in Block 210 to be implemented for the method 200.

Survey datasets 215 can include survey responses to one or more of: current migraine status survey; quality of life survey; trigger survey; pain level survey; medication survey; symptom survey; root cause survey; adherence survey. The surveys can additionally include any other suitable surveys configured to assess migraine and headache patient states (e.g., mental health, lifestyle, pain, mood, motivation, etc.) or therapeutic intervention (e.g., music therapy, physical therapy, medication reduction, etc.). Survey datasets are configured to include quantitative scores, qualitative responses, and any other suitable method of processing to produce an outcome value. Additionally Block 210 is configured to analyze and store survey datasets 215 and modify instructions for the method 200 to optimize user experience.

In continuation of the previous example, Block 210 is configured to analyze patient health history dataset 211 and the feedback dataset 213 according to data collected from historical patient data 110, real time patient data 120, and external data sources 130. Block 210 will process patient health history dataset 211 and patient feedback dataset 213 and store survey responses in patient survey datasets 215 to identify potential patterns and generate survey responses.

For example, Block 210 will process patient health history dataset 211 (e.g., has 10 years of EHR data, female, 5′5″, 160 lbs., married, two children, stay-at-home mom, Middle Eastern ancestry, blood type A−, currently on three medications with a history of eight previously used, daily vitamin regimen consisting of multivitamin and magnesium, 50% of food is home cooked with the other 50% being takeout, minimal physical activity, and poor sleep hygiene) and patient feedback dataset 213 (e.g., high interest for activities with children and low impact exercises for blood type A; neutral feedback for vitamin regimen and nighttime sleep challenge; and negative feedback for reduction of medication consumption). The system will transmit instructions for Block 201 to analyze, develop correlations, and generate instructions to be used by method 200 for delivery of additional survey responses. Additionally, survey responses according to the datasets 211 and 213 may request additional data as to why the patient has negative feedback for reduction of medication or a high interest in low impact exercises. The data obtained in the patient feedback datasets 213 can be further used to train and modify the models within Blocks 301, 401, 501, and 601.

To further the example, if patient responses from the survey dataset 215 include (negative feedback for reduction of medication due to a thyroid removal surgery resulting in lifelong medication adherence; high interest in low impact exercises for weight loss; and high interest to discover new activity to not trigger physical exertion headache), the system will transmit instructions for Block 201 to identify potential correlations for log of use dataset 217. Additionally, the system may transmit survey dataset 215 to Block 651 for analysis by a care team member, and/or any other suitable module for processing.

Additionally, Block 201 is implemented using the patient data collection module 210 and configured to receive and/or collect communication-related data from a patient's mobile communication device with instructions to store data in the log of use dataset 217. In a variation, Block 210 is configured to interface with the patient's mobile computing device (e.g., smartphone, tablet, wearable computing device, vehicle, etc.). In implementing Block 210 to receive log of use dataset 217, the digital therapeutic application will be an active background process on the patient's mobile communication device to gather patient data. Additionally, Block 210 is optimized for automatic data collection of the log of use dataset 217.

Regarding the log of use dataset 217, Block 210 enables the collection of one or more care-team telehealth related data (start time, end time, number of attended sessions, number of missed sessions, etc.), messaging (e.g., chatbot, emails, or peer messages) data (e.g., amount of messages, time associated with sent or received), digital therapeutic content (challenges, exercises, badges earned, lessons watched, lessons listened), and application activity (e.g., time spent on application, marketplace purchases, food logs, assessments, etc.). Block 210 is additionally configured to process log of use datasets 217 with identifiers to classify one or more of the following: intended to be performed once (e.g., lab test, marketplace purchase, therapy, etc.), associated with a particular location (e.g., sanctuary, home, work, away from home, car, at a desk, coffee shop, gym, doctor office, lab testing facility, etc.), with a particular third party (e.g., family, kids, co-workers, friends, church group, small group, etc.), with a regularly occurring event, and/or with a certain type (e.g., relaxing, physical, spiritual, fun, etc.), with a particular time of day (e.g., morning, lunchtime, evening, on a break, less than once a day, daily, less than once a week, etc.), intended to be performed at reoccurring and/or irregular times (e.g., eating anti-inflammatory foods, consuming less gluten, deep breathing, morning stretching, coaching calls, therapy sessions, taking vitamins, etc.), linked with comorbidities (fibromyalgia, obesity, high blood pressure, diabetes, prediabetes, endometriosis, epilepsy, asthma, depression, anxiety, dipolar disorder, panic disorder, etc.) and/or any other suitable health condition; intended to be performed at specified time periods (e.g., medication, taking a walk during a break, closing eyes to avoid eyestrain, eating a snack to avoid fluctuations in blood sugar, scheduling an additional therapy session for increased stress, attending a coaching session for accountability, etc.).

In a continuation of the previous example, Block 210 will generate a modified analysis based on log of use datasets 217, in correlation to patient health history datasets 211, patient feedback datasets 213, and patient survey datasets 215. Block 210 is configured to process patient log of use datasets 217 according to digital therapeutic application usage (e.g., patient has journaled increased anxiety in evening, increased viewership of anxiety-related content, and enrollment into 7-day anxiety breathwork challenge). Additionally, the system 100 is configured to instruct Block 210 to retrieve and/or process external data sources 130 to identifying potential associations with digital communication behavior and patient health history datasets 211, patient feedback datasets 213, patient survey datasets 215, and log of use datasets 217 (e.g., frequent social media usage between 11 pm and 2 am may be a potential link for anxiety, digestive symptoms, poor sleep hygiene, unhappy mental image of current weight, etc.).

In relation to log of use datasets 217, data can be used and processed to generate and/or modify instructions in Block 220 to retrieve or collect symptom map 221, migraine profile 223, and triggers 225. In another variation, Block 201 can further facilitate automatic survey and/or feedback responses to identify potential patient trends between historical patient data 110, real time patient data 120, external data source 130, patient health history dataset 211, log of use dataset 217, symptom map 231, migraine profile 233, triggers 235. Additionally, Block 201 can facilitate automatic retrieval of patient log of use 217 and generate an analysis for potential patient trends between historical patient data 110, real time patient data 120, patient health history dataset 211, symptom map 231, migraine profile 233, and triggers 235.

For example, the patient mobile computing device receives historical patient data 110 (e.g., lab test results and diagnostic images from a specific time period relating to migraine, digestive-related symptoms, and potential IBS), automatically detected user activity from real time patient data 120 (e.g., food trigger survey), patient health history 211 (e.g., patient has poor sleep hygiene, is on 3 medications, and has a BMI of 31%), log of use dataset 217 (e.g., patient takeout consumption increased over the weekend and digital therapeutic app usage increased between the hours of 11 pm and 2 am), symptom map 221 (e.g., patient has experienced migraine with digestive-related symptoms for 5 years and experiences an average of 70% prodrome symptoms), migraine profile 223 (patient has worst migraine on Sunday evening and Monday morning), triggers 235 (e.g., food sensitivity, chemical, and emotional triggers).

### 3.2 Migraine Map Profile Building Module

Block 220 Migraine Map Profile Generating Module processes datasets from at least one of Symptom Map 221, Migraine Profile 223, and Triggers 225.

Block 220 functions to collect and/or retrieve datasets 221, 223, and 225; generate a patient migraine profile; and determine values of one or more migraine and headache health metrics over a time period. Block 220 can additionally or alternatively include datasets from Block 210 or Block 230, and/or datasets from 110, 120, and 130. Block 220 enables the assessment of migraine and headache patterns (e.g., pain, frequency, duration, symptom, trigger, medication use, lifestyle habits, migraine, or headache types, etc.) and can additionally or alternatively predict patient trends toward a future migraine or headache state. Additionally, Block 220 enables the assessment of historic or current migraine and headache status and/or predicts the risk toward triggering a future migraine, headache, or stimuli event at a period of time.

In an example, Block 220 can include a set of digital communications describing a series of lifestyle activities for tension-type headache may be correlated with a decreased risk of migraine. In another example, a series of messages describing increased stress food and grocery shopping can be correlated with a migraine and headache health metric indicating a higher probability of a potential trigger 225; and updating Block 315, Block 325, and Block 335.

In a variation, Block 220 can be based on a survey dataset 215 from a patient's response to a migraine and headache evaluation survey and a log of use dataset 217. For example, patient responses to symptoms, pain location, treatment (e.g., abortive and/or preventative medications), therapeutic intervention (e.g., chiropractic, journaling, breathwork, exercise, stretching, etc.) and/or lifestyle habits (e.g., food, supplements, etc.) can be used in generating the evaluating migraine and headache risk in generating the migraine and headache health metric.

In another example where the risk factors for migraine and headache are evaluated, the number of confirmed risk factors can be transformed into a probability of migraine, headache type, symptom, trigger, onset, pain (type, location, etc.), ER visit, medication, and/or therapeutic intervention.

Additionally, Block 220, is configured to identify and analyze potential correlations between symptoms, treatments, health outcomes, potential missed treatment(s) and/or therapeutic intervention(s), misdiagnosis and/or missed diagnosis, and/or unintended consequences.

In a specific example, a patient attends a scheduled care team appointment (e.g., primary care physician), with symptoms (e.g., nausea, diarrhea, sinus congestion, pain lasting over 72 hours, pain on the left side of the head, and photophobia), and prescriptions (e.g., Zofran for nausea, Flonase for sinus congestion, and high-dose ibuprofen for head pain).

In a variation of the same example, one patient subgroup from the population datasets 231 may have a positive benefit from prescription (e.g., Zofran) for symptom (e.g., nausea). However, another patient and/or patient subgroup from the population datasets 231 may experience side effects (e.g., headache, diarrhea, and worsened headache), unintended consequences (e.g., prescribed additional medication for new symptoms and worsened headache), and/or missed treatment (e.g., nausea triggered by food sensitivity and Vitamin B-12 deficiency).

Additionally, Block 220 functions to monitor patient quality of life status (e.g., retrieved from survey datasets, journaling prompts, log of chatbot messages, etc.) over a specific time period (e.g., one week after initial treatment, etc.), categorize positive and/or negative health outcomes, determine missed treatments, and analyze unintended consequences.

Block 220 includes determining one or more triggers from datasets 110 and 120, and interventions using the datasets 213, 215, and 217.

Symptom map 221, is configured to retrieve and/or collect data from datasets 110, 120, 215, and 217 processed into symptom categories (e.g., migraine, headache, comorbidity, health condition, risk factor, genetics), with identifiers (e.g., organ system, organ, body function, root cause, neurotransmitter, imbalance, deficiency), associations (e.g., food, nutrient, physical symptom of emotional cause), triggers (e.g., emotional, physical, chemical, etc.), and/or any other suitable value or identification to be used by the system.

Migraine profile 223, is configured to retrieve and/or collect data from datasets 110 and 120 and process into any one or more of the following: pain level, frequency (chronic, episodic, intractable, new daily persistent), duration (e.g., minutes, hours, days, months, years, etc.), measures indicative of headache types or other comorbid conditions (anxiety, depression, autoimmune disease, sinusitis, allergy, etc.), physical activity (fitness metrics, pulse rate, blood pressure, etc.) metrics correlated with migraine health (e.g., sleep, stress, stigma, etc.), vital signs, or any other suitable metric relating to migraine and headache health.

Triggers 225 is configured to retrieve and/or collect data from datasets 110, 120, 213, 215, and 217 to be processed in categories and accessed by Block 320. The trigger dataset is optimized to process data in accordance to Block 320. Categories may include dietary, lifestyle habits hormonal, environmental, stressors, physical, sleep, medication, nutritional, chemical, pollutants, genetic and/or any other suitable trigger category as it relates to Block 320.

The system will send instructions to Block 201 to process Block 220 and datasets 221, 223, and 225 to be analyzed in Block 230

### 3.3 Supplementary Data Collection Module

The supplementary data collection module, Block 230, is configured using algorithms from the system 100 to retrieve and evaluate population datasets 231 and supplementary datasets 233. The prepared data can be accessed by the system and distributed across the method 200 to generate an analysis of data to identify potential correlations for improved therapeutic response.

Population datasets 231, can include data gathered from real time patient data 120, patient feedback dataset 213, patient survey dataset 215, log of use database 217, and/or care team 161.

In a variation, Block 230 functions to prepare datasets to determine potential correlations within a group and/or subgroup of the population according to data processed in Blocks 210 and 220. For example, the system identifies 20% of women patients experience migraine, report menstruation as the primary trigger, have increase photophobia and stress prior to migraine pain, and top symptoms include nausea and dizziness. The system will send instructions to Block 230 to process population data to identify any potential trends in one or more of the following population groups or subgroups.

Additionally, Block 201 can analyze data processed by Block 230 and reference log of use data for insights (e.g., application usage, activity, etc.).

In a variation, Block 230 will collect and/or retrieve external data source 132 and 133 to process external IoT data and data from external databases. For example, patient is having photophobia with a level 8 pain migraine. Patient can access the application, access synced devices, and transmit the setting to alter the hue of the lights, turn on the humidifier, turn on music, and/or any other predetermined settings created by patient in accordance to migraine headache type, symptom, trigger, and/or migraine-related comorbidity.

Block 230 is configured to retrieve and/or collect and process one or more of the following datasets 110, 120, 130, 211, 213, 215, 217, 221, 223, and 225 to generate supplementary datasets 233. For example, supplementary datasets can retrieve, analyze, and/or process datasets into application usage (e.g., chatbot usage log, social group datasets; therapeutic intervention datasets); content characteristics (e.g., chatbot content, message content, post content, comment content, media content, profile content, etc.); device sensor datasets (e.g., mobility supplementary datasets; etc.); device usage information (e.g., screen usage information, physical movement of the mobile device, etc.); lab test result information (e.g., symptom-lab result connection with low Vitamin D, high fasting glucose for blood sugar headache, imbalance in progesterone as a trigger for menstrual migraine), and/or any other suitable lab test characteristics; light sensor information (e.g., measuring amount of user light exposure, etc.); location information (e.g., environmental datasets, GPS sensor information,), and/or any other suitable location characteristics; marketplace usage information and quantitative characteristic (e.g., clicks, views, purchases, bookmarks, shares, etc.); medical device data (CT scan, x-ray, MRI, EEG, EKG, ECG, EDG, ultrasound, facial recognition, etc.,); mobile computing device sensor information can include recorded biosensors and/or biosensors (e.g., vitals, heart rate, blood pressure, biofeedback signals, EEG signals, blood sugar levels, body fat percentage, metabolic age, etc.); physical activity information (e.g., inertial sensor data, etc.), and/or any other supplementary physical activity characteristics connected by supported devices; and survey datasets (e.g., health history, symptom map, migraine profile, headache types, behavioral health).

System 100 can send instructions from Block 201 to Block 301 to use data in the generation of migraine headache analyzer models. Additionally, the system and method can transmit datasets from Block 201 in any other suitable manner.

### 4. Migraine Headache Analyzer

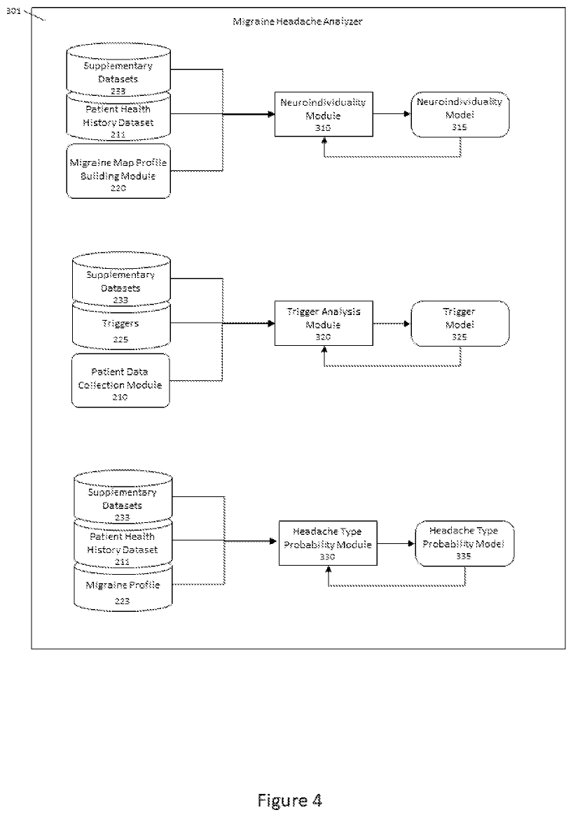

As shown in FIG. 4, the migraine headache analyzer, Block 301, consists of Neuroindividuality module 310, Neuroindividuality model 315, trigger analysis module 320, trigger model 325, headache type probability module 330, and headache type probability model 335. Block 301 is configured to process many datasets from Block 201 and prepare data to be used by the system 100, throughout the method 200, to generate therapeutic lifestyle interventions according to the patient's migraine headache profile.

The exemplary migraine headache analyzer shown in FIG. 4 comprises at least the following:

### 4.1 Neuroindividuality Module

Block 310, Neuroindividuality model, functions to use one or more machine learning techniques, data mining, or statistical approaches to generate increasingly accurate models regarding a patient's migraine and headache health status.

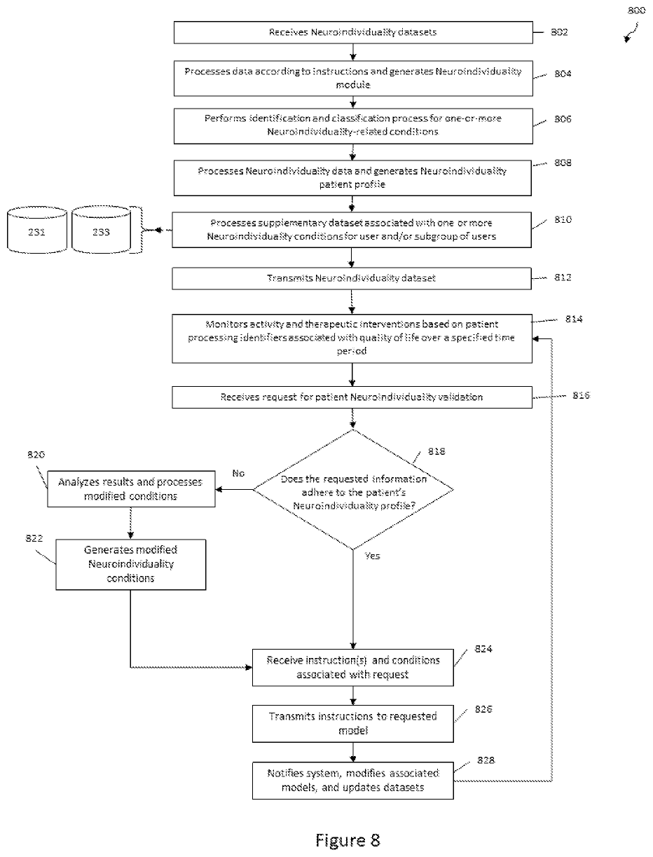

As shown in FIG. 8, an example procedure 800 of the Neuroindividuality method for Block 310 will include receives Neuroindividuality datasets 802; processes data according to instructions and generates Neuroindividuality module 804; performs identification and classification process for one-or-more Neuroindividuality-related conditions 806; processes Neuroindividuality data and generates Neuroindividuality patient profile 808; processes supplementary dataset associated with one or more Neuroindividuality conditions for user and/or subgroup of users 810; transmits Neuroindividuality dataset 812; monitors activity and therapeutic interventions based on patient processing identifiers associated with quality of life over a specified time period 814; receives request for patient Neuroindividuality validation 816; does the requested information adhere to the patient's Neuroindividuality profile? 818; with a no response - analyzes results and process modified conditions 820; generates modified Neuroindividuality conditions 822; receive instruction(s) and conditions associated with request 824; transmits instructions to requested model 826; notifies system, modifies associated models, and updates datasets 828.

In an example, Block 310 can include applying algorithms to patient migraine and headache data from a log of use dataset 217 and a supplementary dataset 233 to display predicted patient migraine and headache health data. Data can be labeled with a Neuroindividuality classification risk value, health metric scores, therapeutic intervention, lifestyle habit, comorbid risk factor, or any other suitable label pertaining to a migraine and headache patient's Neuroindividuality. Block 310 is implemented at the system 100 to process data from one or more of the datasets and/or instructions found in Block 201. The patient data may be stored locally or remotely and is processed and retrieved through the system 100.

When generating Block 310, patient data from one or more datasets from Block 201 will provide a set of corresponding time periods to determine parameters in association with a patient's critical health metrics and migraine and headache values. In variations, correlations of time periods and patient data can determine the migraine and headache health status in relation to the patient's migraine phase and the association between active and passive data.

Another variation, Block 310, can employ a predictive modeling engine to assess historical patient data, allopathic treatment regimens, integrative therapy interventions, best-in-class neurological and migraine guidelines, lifestyle habits, and stress levels. For example, a patient may be prescribed an abortive and preventative medication, as well as Botox, has poor sleep hygiene, consumes inflammatory foods, and is regularly stressed about work and finances. The resulting predictive information can be displayed in the context of charts and graphs highlighting the historical data and predictive Neuroindividuality data. The embodiment of the present invention can include identifying increased stress with tension-type headache and non-drug intervention to potentially reduce current symptoms and pain levels, and furthermore, mitigate future onset to improve quality of life.

In another variation, Block 310 monitors symptoms and medication adherence to determine trends within headache frequency and duration, pain location and levels, and symptom patterns to predict the potential risk of medication-overuse headache, in conjunction with lifestyle habits. Based on the risk classification, Block 310 can display charts, graphs, or alerts to care team physicians for monitoring pharmaceutical intervention, and if required, modifying current prescriptions.

The Neuroindividuality model, Block 315, is designed to generate predictive models in any suitable manner that define labels, values, relationships, and/or outputs, over a specified time period, to demonstrate a positive and/or negative connection between trigger, migraine headache type, intervention, and quality of life. Blocks 310 and 315 are designed as a feedback loop to be accessed by the system and method.

### 4.2 Trigger Analysis Module

Trigger analysis module, Block 320, is based on one or more of supplementary datasets, trigger datasets, and datasets in Block 210. Block 320 functions to determine relevant migraine and headache triggers.

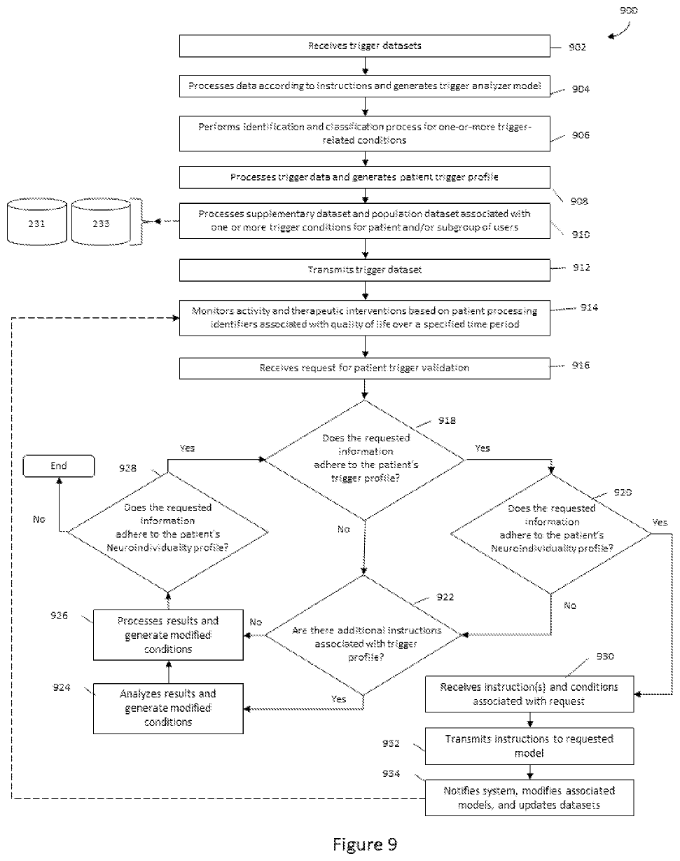

As shown in FIG. 9, an example procedure 900 of the trigger analysis method for Block 320 will include receives trigger datasets 902; processes data according to instructions and generates trigger analyzer model 904; performs identification and classification process for one-or-more trigger-related conditions 906; processes trigger data and generates patient trigger profile 908; processes supplementary dataset and population dataset associated with one or more trigger conditions for patient and/or subgroup of users 910; transmits trigger dataset 912; monitors activity and therapeutic interventions based on patient processing identifiers associated with quality of life over a specified time period 914; receives request for patient trigger validation 916; does the requested information adhere to the patient's trigger profile? 918; does the requested information adhere to the patient's Neuroindividuality profile? 920; are there additional instructions associated with trigger profile? 922; analyzes results and generate modified conditions 924; processes results and generate modified conditions 926; does the requested information adhere to the patient's Neuroindividuality profile? 928; receives instruction(s) and conditions associated with request 930; transmits instructions to requested model 932; notifies system, modifies associated models, and updates datasets 934.

In an example, Block 320 may include dietary (eating habits; food lists; processed foods, nitrates, sulfates, msg, soy, corn, shellfish, soda pop, artificial sweeteners, high-fructose corn syrup, high-histamine, inflammatory, tyramine, cheese, conventional meat; cooking habits; food allergy foods; food sensitivity foods; etc.), lifestyle habits (physical activity, dehydration, stress management, self-care, social media habits, etc.), hormonal (hormonal imbalance symptoms, hormonal imbalance lifestyle factors, endocrine disruptors, cortisol imbalance information, medication correlation information, etc.), environmental (allergens, barometric pressure shifts, altitude shifts, seasonal change, food correlation information, etc.), stressors (relational, lack of support, work, psychosocial, financial, world view beliefs, etc.), physical (accidents, injury, brain trauma, exertion-type headache, etc.), sleep (changes in sleep patterns, dreams, etc.), emotional and spiritual (repressed emotions, cognitive distortions, self-criticism, self-esteem, perfectionism, information overload, trauma, resentment, worry, guilt, shame, etc.), medication (usage statistics, risk correlation information, FDA recall, etc.), nutritional (symptom deficiency information, supplements, eating habits; etc.), chemical (consumer products, demographic information, allergy symptom correlation, etc.), pollutants (herbicides, insecticides, heavy metals, radiation, light, etc.), genetic (familial migraine and headache history, genetic predispositions, etc.), sensory sensitivity (photophobia, phonophobia, smells, etc.), organ systems (digestive, musculoskeletal, immune, etc.), illness (viral, bacterial, fungal, etc.), mental health (anxiety, depression, panic attacks, etc.), and migraine-associated comorbid factors (anxiety, depression, obesity, autoimmune disorders, neurological conditions, allergy, sinusitis, etc.).

Block 320 can include supplemental user information (e.g., lab test results, diagnostic test results, past medical history, eating habits, sleeping patterns, medication adherence statistics, patient profiles, social media profiles, care team diagnostic data, or any other suitable supplemental user information). Additionally, supplemental data and datasets collected in Block 201 can be used in ranking triggers by type, category, demographic, socioeconomic, geographic, gender, therapeutic intervention, content, lifestyle intervention, lab test, time period, or any other suitable criteria. In a variation, Block 320 can include the identification of a subset of users associated with migraine and headache triggers, classified and/or ranked by a supplemental dataset. For example, a subset of users with a specific migraine and headache type, trigger, and comorbid factor may be more favorable to non-drug lifestyle interventions.

Block 320 is preferably based on datasets collected in Block 210. In a variation, Block 320, can be determined based on a log of use dataset. The method 200 can include identifying correlations between a population subset (e.g., women with menstrual migraine), log of use dataset (e.g., medication adherence, food, and stress management activity), and one or more migraine and headache-related conditions (e.g., the correlation between stress, eating habits, lifestyle activity, and migraine frequency); determining a migraine and headache trigger analysis based on correlations and log of use dataset, promoting a lifestyle recommendation based on the migraine and headache trigger analysis; and updating the migraine and headache trigger analysis from an updated log of use (e.g., determining the migraine and headache trigger based on improved quality of life in response to lifestyle, therapeutic intervention, pharmacological agent, non-drug therapy, etc.).

The trigger model, Block 325, is designed to generate predictive models in any suitable manner that define labels, values, relationships, and/or outputs, over a specified time period, to demonstrate a positive and/or negative connection between trigger, migraine headache type, Neuroindividuality, intervention, and quality of life. Blocks 320 and 325 are designed as a feedback loop to be accessed by the system and method.

### 4.3 Headache Type Probability Module

The headache type probability module, Block 330, functions to generate an analysis of the patient's migraine and headache profile, status, and/or characteristics to be used in the delivery of lifestyle therapeutic interventions.

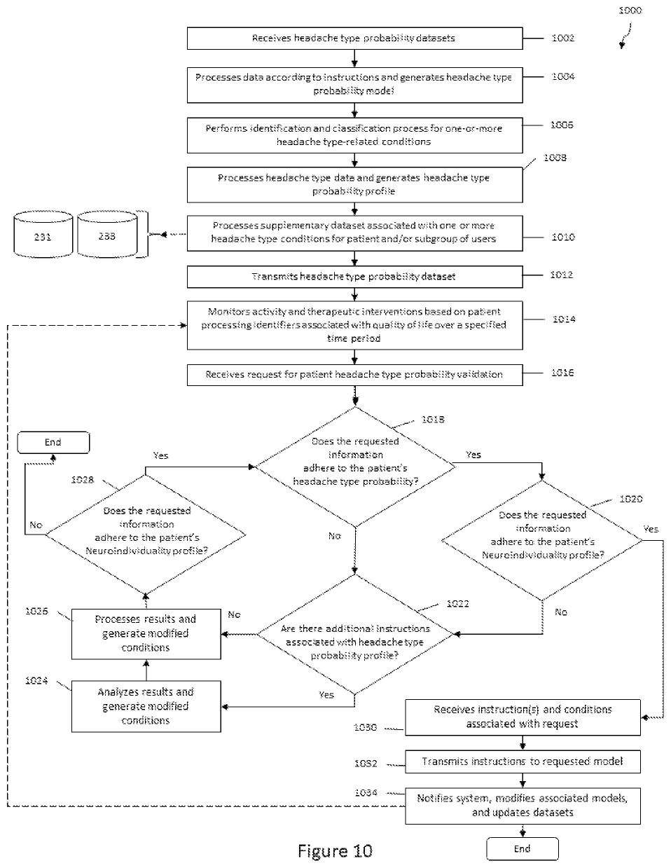

As shown in FIG. 10, an example procedure 1000 of the headache type probability method for Block 330 will include receives headache type probability datasets 1002; processes data according to instructions and generates headache type probability model 1004; performs identification and classification process for one-or-more headache type-related conditions 1006; processes headache type data and generates headache type probability profile 1008; processes supplementary dataset associated with one or more headache type conditions for patient and/or subgroup of users 1010; transmits headache type probability dataset 1012; monitors activity and therapeutic interventions based on patient processing identifiers associated with quality of life over a specified time period 1014; receives request for patient headache type probability validation 1016; does the requested information adhere to the patient's headache type probability? 1018; does the requested information adhere to the patient's Neuroindividuality profile? 1020; are there additional instructions associated with headache type probability profile? 1022; analyzes results and generate modified conditions 1024; processes results and generate modified conditions 1026; does the requested information adhere to the patient's Neuroindividuality profile? 1028; receives instruction(s) and conditions associated with request 1030; transmits instructions to requested model 1032; notifies system, modifies associated models, and updates datasets 1034.

In an example, Block 330 can include one or more of the supplemental datasets (e.g., familial migraine and headache history, etc.); user migraine and headache lifestyle behavior based on datasets collected in Block 210; correlations between lifestyle, symptoms, and triggers; neurological and migraine-associated comorbid factor medical information derived from external sources (e.g., American Neurological Association, American Migraine Association, American Diabetes Foundation, Asthma and Allergy Foundation of America, American Academy of Allergy, Asthma, and Immunology, Allergy and Sinus, American Psychological Association, health insurance companies, regulatory agencies, pharmaceutical companies, etc.); non-drug therapeutic intervention information derived from databases and resources (e.g., consumer product companies, social groups, etc.); user population therapeutic and non-drug interventions; and/or any other suitable dataset used in determining the migraine and headache type probability analysis.

Additionally, Block 330 can include retrieving survey, feedback and/or log of use datasets related to therapeutic intervention (e.g., pharmacological, non-drug, or lifestyle), diagnosis, and/or recommendation (e.g., content, social interaction, product, care team member, etc.).

In a variation, Block 330 can use data from Block 320 to determine the correlation of behaviors to headache types, pain levels, and onset patterns. Additionally, the system can process and analyze data from Block 320 to modify Block 315.

In another variation, Block 330 can include ranking or classification of users (e.g., patients associated with a particular migraine and/or headache type; patients with migraine and/or headache type by demographic, geographic or socioeconomic status; patients associated with frequency, duration, or pain level; etc.) for determining potential therapeutic, pharmacological, and/or non-drug interventions. The system can process data and instructions to modify Block 335.

For example, method 200 can include identifying a population subset of users associated with migraine, headache, or migraine-associated comorbidities. Classifying and/or ranking users can be based on type, diagnosis length, intervention efficacy, readiness, patient monitoring, and datasets collected in Block 201 (e.g., health history, symptom map, migraine profile, headache types, logs of use, communication, supplementary, etc.).

Regarding Block 335, the model can be trained and updated based on log of use dataset. For example, the method 200 can include identifying correlations between quality-of-life identifiers (duration, frequency, pain levels, missed workdays, etc.), communication behavior (care team, chat, social, messages, etc.), and survey data (migraine and headache tracking assessment); determining the migraine headache type analysis for a user according to correlations within the log of use dataset; delivering a migraine and headache therapeutic intervention according to the datasets 213, 215, and 217; and modifying Block 335 based on correlations and the log of use dataset (e.g., determining an improvement in migraine with aura and photophobia based on an increase in therapeutic lifestyle activity and digital quality of life monitoring; etc.).

In another variation, Block 335, can be modified according to supplementary datasets. For example, the method 200 can include identifying a response to a promoted food product based on the log of use dataset and supplementary dataset (e.g., identifying a correlation between sinus congestion and food during a therapeutic monitoring time period) and updating Blocks 315 and 325 (e.g., food, sinus congestion, and headache onset).

In another variation, Block 335 can be modified in relation to communication data (e.g., users interacting with users, users interacting with a chatbot, and/or users interacting with care team members, etc.). For example, Block 330 can include updating Block 335 according to user social interaction (e.g., communications referencing headache type, symptom, triggers, pain levels, migraine stigma, failed and/or successful interventions, etc.). In another example, Block 335, can reference a communication log of use with the chatbot (e.g., communications suggesting and/or asking for help with a headache type, symptom, trigger, etc.). In a specific example, Block 330, can be based on patient interactions with a care team member (e.g., patient suggests a connection between food, sinus congestion, and headache pain; historical treatments; summary of headache duration and frequency after recognized trigger; etc.).

Block 330 can include updating Blocks 315, 325, and 335. In an example, the method 200 can include identifying a diagnosis of migraine and headache type and migraine-associated comorbid factor; therapeutic lifestyle interventions and pharmacological modifications; presenting diagnosis and intervention to a patient's care team (e.g., neurologist, dietician, psychologist, and health coach); receiving neurologist verification and validation of care; generating a new Neuroindividuality model; generating an updated trigger probability analysis; monitoring user activity over a time period; and providing digital content.

In a specific example, the method 200 can include identifying a diagnosis of migraine type (e.g., chronic transforming into new daily persistent), headache type (e.g., tension-type), and migraine-associated comorbid factor (e.g., anxiety, allergy, and sinusitis); determining lifestyle interventions (e.g., modifying diet and nutrition, breathwork exercises and stretching for increased tension, change in morning routine, lab tests for food allergy and nutrient levels, and cognitive behavioral therapy techniques); modifying pharmacological interventions (e.g., changing the medication and/or medication dosage from an abortive migraine medication or a CGRP); presenting diagnosis and intervention to care team (e.g., neurologist modifying prescription and overseeing treatment protocols, dietician modifying meal plans and analyzing lab results, psychologist facilitating cognitive behavioral therapy techniques, and health coach assisting with accountability in lifestyle change); and providing digital content (articles, interviews, medical information, drug information, medical journals, etc. based on updated migraine and headache type probability analysis).

In another variation, the system can send instructions to Block 335 to monitor patient migraine phases, triggers, treatment protocols, therapeutic interventions, time periods, patient readiness, and quality of life. Additionally, Blocks 210, 220, 315, 325, and 335 can work together to generate a therapeutic intervention for a patient during the onset of a migraine and/or headache; therefore, modify Blocks 415 and 525.

In an example, a patient diagnosed with headache type (tension-type headache), symptom (extreme tightness in the neck and shoulders), pain level (high 8), and received a therapeutic recommendation (chiropractic care), the method 200 can include determining the likelihood of therapeutic implementation (location, insurance status, cost, provider experience, etc.), and quality of life (intervention improves or worsens migraine and/or headache pain, frequency, duration, etc.) according to the log of use, survey (e.g., migraine and headache assessment, symptom assessment, trigger assessment, etc.), and supplementary dataset.

In a more specific example, a patient diagnosed with tension-type headache, extreme tightness in the neck and shoulders, and high pain, has a worsened migraine and headache pain due to chiropractic care. Block 335 can transmit instructions to the system and modify the Block 415 and 525 to execute Block 510 a second therapeutic intervention. Alternatively, updating the therapeutic intervention and migraine and headache type probability analysis can result in identifying the potential root cause of the tension-type headache and extreme tightness in the neck and shoulders (e.g., stress, posture, emotional well-being, mental health, eye strain, too much computer work, lack of physical exercise, lack of stretching, musculoskeletal causes, etc.); determining methods of communication (e.g., chatbot prompts, care team interactions, content distribution, survey questions, assessments, etc.); generating a comparison between migraine and headache health metrics, migraine and headache triggers, and migraine and headache types; generating an updated Neuroindividuality model; and generating an updated model for therapeutic intervention.

Block 325 is designed to generate predictive models in any suitable manner that define headache type labels, values, relationships, and/or outputs, over a specified time period, to demonstrate a positive and/or negative connection between intervention and quality of life. Blocks 330 and 335 are designed as a feedback loop to be accessed by the system and method.

### 5. Intervention Predictor

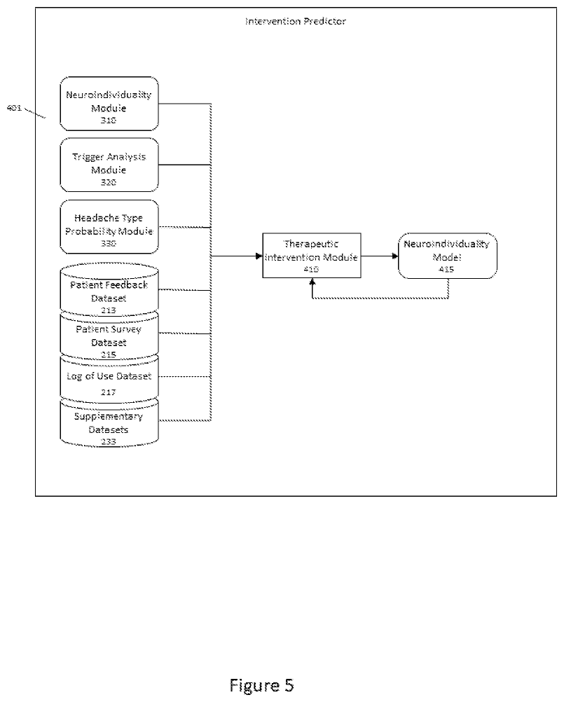

As shown in FIG. 5, the intervention predictor, Block 401 functions to perform identification and classification processes for one or more Neuroindividuality-intervention conditions from Blocks 410 and 415. Block 401 is configured to process many datasets from Block 201 and 301 and prepare data to be used by the system 100, throughout the method 200, to select therapeutic lifestyle interventions according to the patient's migraine headache profile.

### 5.1 Therapeutic Intervention Module

As shown in FIG. 5. the therapeutic intervention module, Block 410 is configured to collect and/or retrieve datasets from 213, 215, 217, 233, 310, 320, and 330 and function to identify migraine and headache type and migraine-associated comorbid therapeutic interventions with a patient. Block 410 is designed to continuously update Block 415 and operate as a feedback loop.

Block 410 may use multiple variables (e.g., physical activity, lifestyle habits, migraine and headache days per month, medication adherence, total migraine and headache symptoms, total migraine and headache triggers, etc.) to predict which patients are most likely to implement therapeutic intervention and achieve an outcome (e.g., improved or worsened migraine and/or headache state). Patients can be categorized by migraine and headache type (e.g., migraine, migraine with aura, tension-type, new daily persistent headache, medication-overuse headache, vestibular migraine, etc.), frequency (episodic, chronic, intractable, etc.), comorbid factors (e.g., allergy, anxiety, autoimmune disorder, depression, epilepsy, hypertension, obesity, sinusitis, etc.), length of time from initial diagnosis, successful and/or failed treatments or therapies, and/or any other suitable categorization method.

The term, therapeutic intervention, can include any one or more of: migraine and headache notification (e.g., automated alerts based on current migraine and headache health status, etc.);

lifestyle interventions (e.g., physical activity, stretching, breathing, morning and/or evening routine modification, meal planning, journaling exercises, etc.); functional medical interventions (e.g., acupuncture, chiropractor, osteopathic, Ayurveda, Traditional Chinese Medicine, physical therapy, music therapy, massage, etc.) care-team member interventions (e.g., messaging dietician and/or nutritionist after identifying potential food trigger, scheduling an additional session with mental health professional to process migraine stigma, interacting with neurologist to coordinate MRI after sudden change in migraine and headache status, contacting care manager to schedule labs, etc.); nutritional interventions (e.g., increased magnesium supplementation, modification of supplementation regimen based on low Vitamin D levels, modification of meal planning according to food sensitivity lab results, etc.); medication modification (e.g., medication triggering potential medication-overuse headache, etc.); and/or other suitable types of migraine, headache, and migraine-associated comorbid therapeutic interventions.

Regarding Block 410, a migraine and headache therapeutic intervention is configured to improve at least one or more migraine and headache health metrics from Blocks 201 and 301. The therapeutic intervention can include lifestyle recommendations, targeted activity, alerts, challenges, exercises, health professional recommendations, migraine trigger modifications, and/or any other suitable migraine, headache, and migraine-associated comorbid therapeutic intervention. Determining the therapeutic intervention can be delivered across all migraine phases (prodrome, aura, pain, and postdrome) and at any suitable time period.

Block 415 is designed to generate predictive models in any suitable manner that define headache type labels, values, relationships, and/or outputs, over a specified time period, to demonstrate a positive and/or negative connection between intervention and quality of life. Blocks 410 and 415 are designed as a feedback loop to be accessed by the system and method.

Additionally, datasets and instructions from Blocks 410 and 415 can be used in Block 201, 301, 501, 601, and 651 to optimize patient response to therapeutic interventions.

### 6. Lifestyle Intervention Module

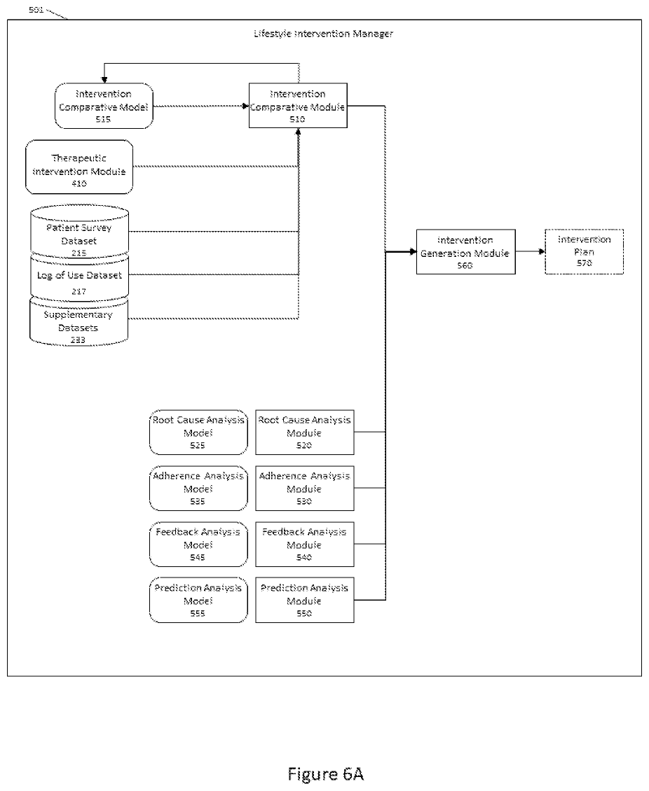

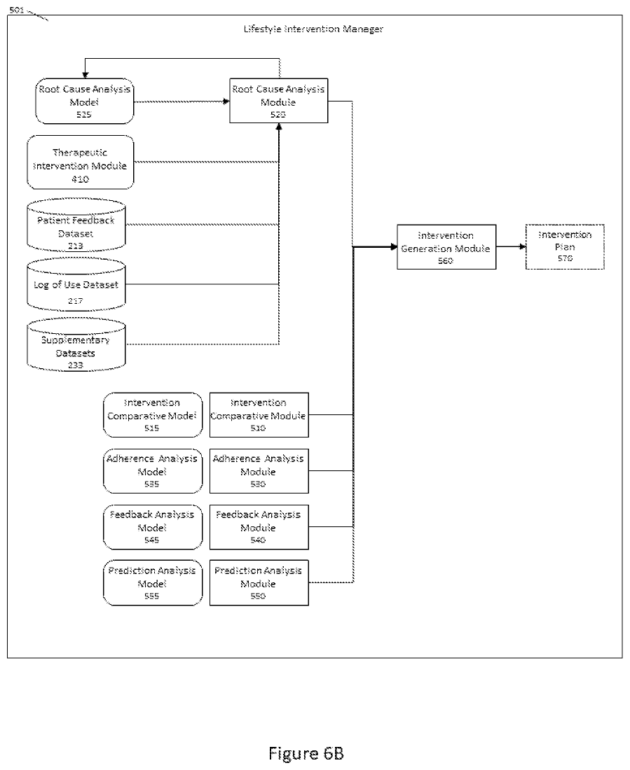

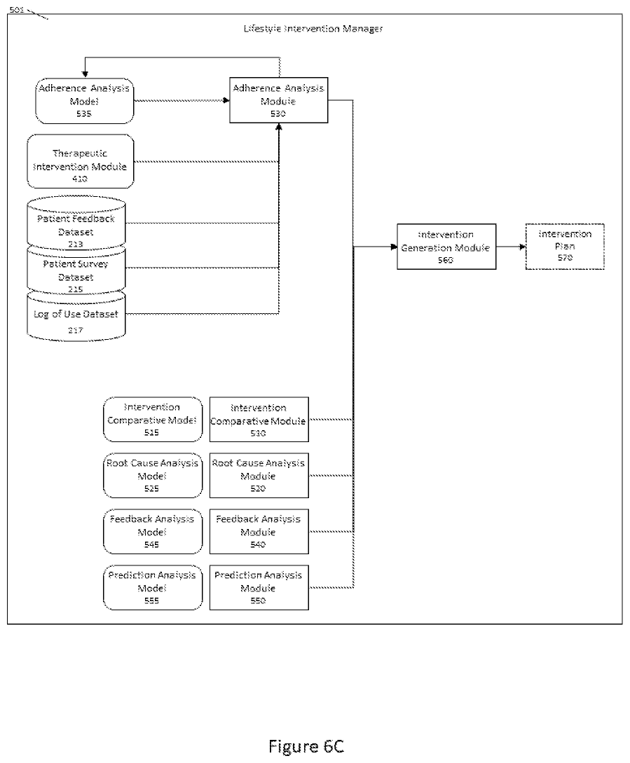

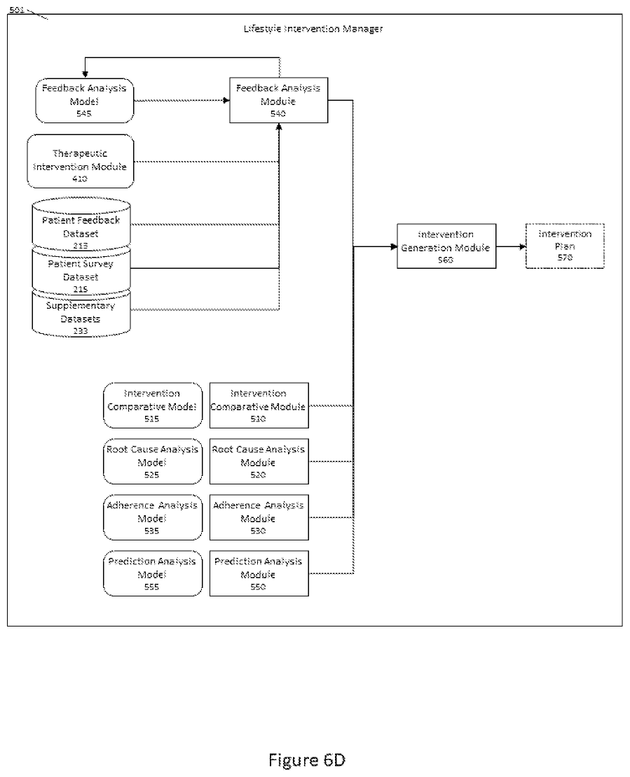

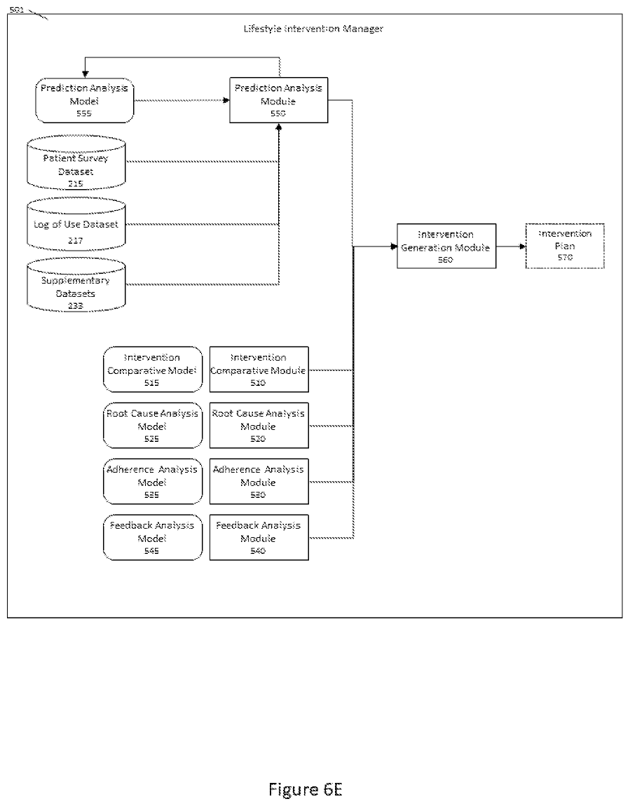

As shown in FIGS. 6A-6E, the lifestyle intervention module, Block 501, consists of intervention comparative module 510, intervention comparative model 515, root cause analysis module 520, root cause analysis model 525, adherence analysis module 530, adherence analysis model 635, feedback analysis module 540, feedback analysis model 545, prediction analysis module 550, prediction analysis model 555, intervention generation module 560, and intervention plan 570. Block 501 is configured to process many datasets from Block 201, 301, and 401 and prepare data to be used by the system 100, throughout the method 200, to generate optimized therapeutic lifestyle interventions according to the patient's migraine and headache profile.

### 6.1 Intervention Comparative Module

As shown in FIG. 6A, the intervention comparative module, Block 510, is configured to compare one or of patient survey, log of use, supplementary, and therapeutic intervention datasets to determine a migraine, headache, and/or migraine-associated comorbid therapeutic intervention; and modify the intervention comparative model, Block 515. For example, a therapeutic intervention (e.g., taking a break and engaging in a short breathwork exercise) can be based on a patient's migraine and headache type (e.g., vestibular migraine), symptom (e.g., dizziness), migraine-associated comorbidity (e.g., anxiety), triggers (e.g., increased stress, change in sleep, car problems), and determined from log of use, supplementary, and/or survey datasets (e.g., migraine and headache status assessment suggesting increased dizziness and stress).

As shown in FIG. 11, an example procedure 1100 of the therapeutic comparative module method for Block 510 will include receives therapeutic comparison datasets 1102; processes data according to instructions and generates model 1104; generates a correlation between intervention values derived from the log of use dataset, survey dataset, and supplementary dataset 1106; transforms the dataset into an analysis of current patient quality of life, historic patient data, and therapeutic intervention associated with a specific time period 1108; performs identification and classification process for one or more Neuroindividuality-therapeutic conditions 1110; processes data and generates comparison between intervention, Neuroindividuality, and quality of life 1112; processes supplementary dataset associated with one or more patient root cause attributes and/or subgroup of users 1114; transmits instructions to requested source 1116; monitors activity and therapeutic interventions based on patient processing identifiers associated with quality of life over a specified time period 1118; receives request to generate comparative model 1120.

Alternatively, Block 510 can identify significant changes in migraine and headache health patterns to automatically promote an intervention prompt requiring immediate medical attention based on datasets.

For example, the patient survey dataset identifies changes in migraine and headache symptoms to identify a potential thunderclap headache. Digital communications are modified to prompt an alert with additional questions. If patient response exceeds values of migraine and headache type, an automated message will be generated for the patient to engage in immediate medical attention. The system can additionally or alternatively send a prompt for the patient to receive ambulatory services.

In variations, Block 510 can process one or more datasets from the Blocks 201, 301, and 401 to generate the comparative model. Datasets can include at least one of active data (e.g., health history dataset, symptom map dataset, migraine profile dataset, headache types dataset), passive data (e.g., digital application behaviors from log of use dataset; and communication datasets from audio, text, and/or chatbot conversations; supplementary dataset over a time period. Block 510 is configured to process data and monitor the efficacy of migraine, headache, and migraine-associated comorbid therapeutic intervention(s) for a patient or subgroup of the patient population over a specified time period.

Block 510 is additionally configured to generate first, second, third, fourth, etc. comparison models between survey response datasets 215, trigger analysis 320, headache type probability 330, root cause analysis 520, feedback analysis 540, adherence analysis 550, and therapeutic prediction analysis 550. Values and scores are compared to the therapeutic intervention to generate a new comparative model. Comparisons generated in Block 510 function to identify and analyze patient status for one or more of migraine and headache type, migraine-associated comorbid factor, symptom, trigger (e.g., increase or decrease in frequency, duration, or pain), and/or any other suitable patient category.

Examples of Block 510 can monitor and/or facilitate the identification of one or more of survey responses exceeding a pre-determined critical score over a specified time period (e.g., total migraine days increased 200% over the past 90 days, pain levels have decreased 50% over the past two weeks, digestive-related symptoms went from 10 on day one to three over 180 days, changes in sleep patterns result in a 90% likelihood of migraine trigger, failure to engage in breathwork activity prior to the stressful event has an 80% likelihood of triggering a tension-type headache, etc.).

In another variation, Block 510 is configured to identify and define patterns in one or more of passive datasets and supplementary datasets. For example, the comparative model can be defined according to patient's passive data (e.g., friends believe patient is not that sick, family believes patient is making up the migraine, and patient's current neurologist is out of treatment recommendations), active data (e.g., migraine assessment data reporting an increase in tension-type headache, anxiety, nausea, vomiting, digestive discomfort, tightness in neck and shoulders, photophobia, phonophobia, dizziness, and changes in sleep patterns), and historical data (e.g., time periods between passive and active data, and/or any other suitable structure).

The intervention comparative model, Block 515, is designed to generate predictive models in any suitable manner that define labels, values, relationships, and/or outputs, over a specified time period, to demonstrate a positive and/or negative connection between intervention and quality of life. Blocks 510 and 515 are designed as a feedback loop to be accessed by the system and method.

### 6.2 Root Cause Analysis Module

As shown in FIG. 6B, Block 520 is generated based on at least one dataset from Block 310 and dataset 225. Additionally, Block 520 can automatically gather and/or collect data from 110, 120, 130, 213, 215, 217, 231, 233, 310, 320, 330, 410, and 415 to optimize Block 525, root cause analysis model. Block 520 is configured to process a therapeutic intervention and a migraine and headache trigger according to a patient's migraine and headache type, symptom, and/or migraine-associated comorbidity. Block 520 functions to analyze improvement in quality of life over a specified time period.

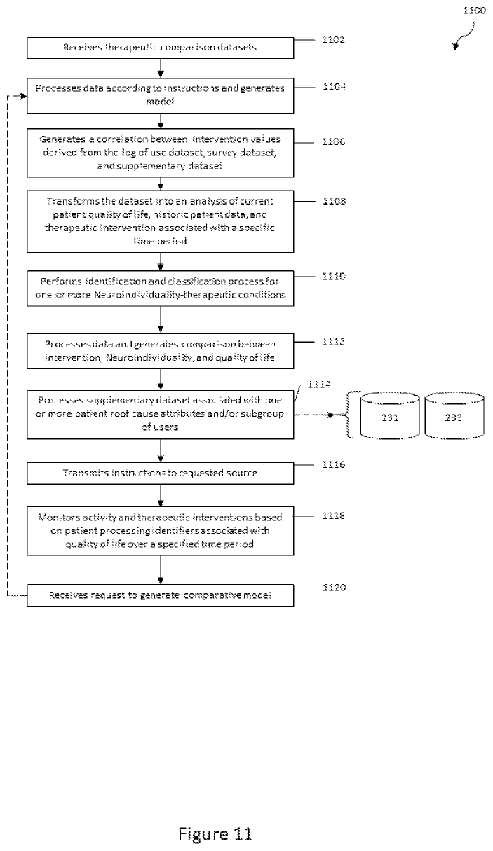

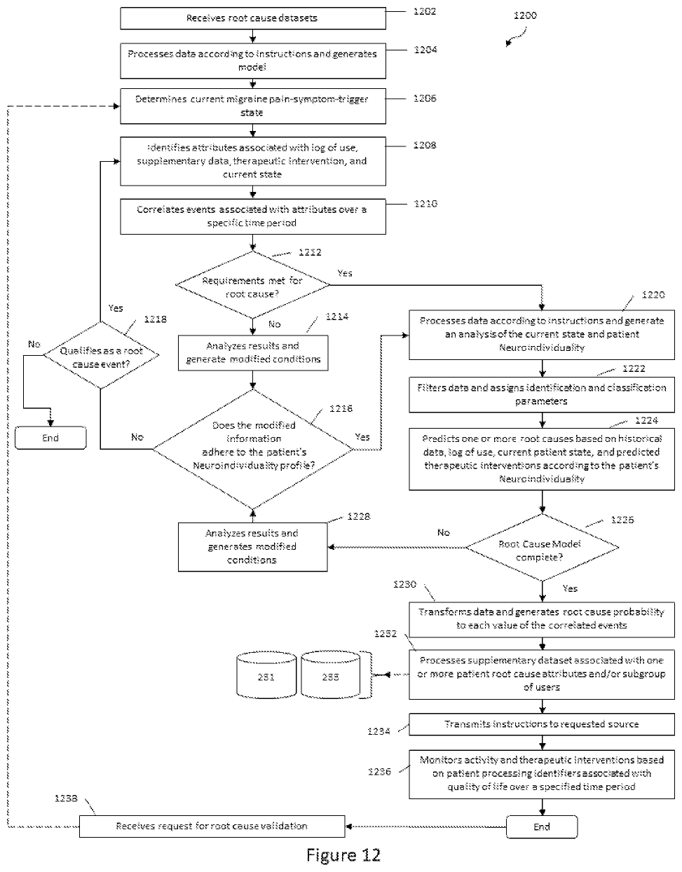

As shown in FIG. 12, an example procedure 1200 of the root cause analysis method for Block 520 will include receives root cause datasets 1202; processes data according to instructions and generates model 1204; determines current migraine pain-symptom-trigger state 1206; identifies attributes associated with log of use, supplementary data, therapeutic intervention, and current state 1208; correlates events associated with attributes over a specific time period 1210; requirements met for root cause? 1212; analyzes results and generate modified conditions from no answer 1214; Does the modified information adhere to the patient's Neuroindividuality profile? 1216; qualifies as a root cause event? 1218; processes data according to instructions and generate an analysis of the current state and patient Neuroindividuality 1220; filters data and assigns identification and classification parameters 1222; predicts one or more root causes based on historical data, log of use, current patient state, and predicted therapeutic interventions according to the patient's Neuroindividuality 1224; Root Cause Model complete? 1226 analyzes results from no answer and generates modified conditions 1228; transforms data and generates root cause probability to each value of the correlated events 1230; processes supplementary dataset associated with one or more patient root cause attributes and/or subgroup of users 1232; transmits instructions to requested source 1234; monitors activity and therapeutic interventions based on patient processing identifiers associated with quality of life over a specified time period 1236; receives request for root cause validation 1238.

In an example, Block 520 may analyze datasets from Blocks 210 and 220 to identify health history information (e.g., height, weight, past and/or current medications, sleep habits, physical activity, blood type, lab results, etc.), symptom map (e.g., migraine symptoms, headache symptoms, migraine-associated comorbidity symptoms, migraine phases symptoms, etc.), migraine profile (e.g., diagnostic history, familial migraine history, pain days per month, average pain levels, etc.), migraine and headache types (e.g., migraine, migraine with aura, tension-type headache, medication-overuse headache, etc.), supplementary (e.g., medication interactions, over-the-counter drug information, food triggers, therapeutic interventions, migraine-associated comorbidities treatment plans, etc.), and communications (e.g., chatbot logs, text messages, social group interactions, etc.).

Additionally, Block 520 can include improvement categories (e.g., quality of life, activity levels, migraine and headache metric, migraine and headache type, migraine-associated comorbid factors, symptoms, triggers, therapeutic intervention, etc.) and/or any other suitable categories used to demonstrate improvement over a specified time period.

Additionally, Block 520 can include datasets demonstrating a negative outcome based on patient survey datasets. For example, an active survey dataset prompted after the use of a new medication identifies associated side effect symptoms. In another example, the patient reports increased frustration, sadness, isolation, and hopelessness after receiving another negative MRI result. Furthermore, Block 520 is configured to monitor patient activity for a continuous time period to determine if the negative MRI results and negative emotions create a correlation to future symptoms, triggers, and/or headache types.

In relation to FIGS. 2-3, Block 520 may transmit instructions to the system to modify Blocks 201 and 301 to monitor correlations between two time periods and therapeutic interventions. For example, the first time period and therapeutic intervention may include symptom (e.g., sinus congestion), migraine phase (e.g., food cravings and prodrome phase, and nasal congestion and headache phase), migraine-associated comorbidity (e.g., acute sinusitis), treatment (e.g., nasal decongestant), log of use dataset (e.g., patient ate a cheese omelet for breakfast and a sandwich for lunch), and survey dataset (e.g., nasal decongestant did not work, migraine pain consistent, and patient uses additional abortive medication).

In a variation, for the second time period with similar root cause symptom (e.g., sinus congestion), migraine phase (e.g., food cravings and prodrome phase, and nasal congestion and headache phase), migraine-associated comorbidity (e.g., acute sinusitis), the system can process data prompt a patient to adjust food intake and substitute for less sinus-inflammatory foods. For example, the patient reports a positive post-intervention survey dataset (e.g., minimal nasal congestion, no food cravings, did not experience sinus congestion, no migraine headache and sinus pressure) based on therapeutic intervention (e.g., fruit smoothie without dairy for breakfast and chicken soup with salad for lunch). Block 520 can generate labels, values, charts, and graphs conveying the patient outcome. Based upon patient survey data, Block 520 can generate correlations between lifestyle activity (e.g., consumption of sinus inflammatory foods), therapeutic intervention (e.g., food and ingredient substitution), migraine and headache type and migraine-associated comorbidity (e.g., migraine, sinus headache, and acute sinusitis).

Additionally, Block 520 is configured to promote therapeutic interventions to improve the efficiency of medical treatments, increase access to care, and decrease risk of potential migraine misdiagnosis.

Additionally, therapeutic recommendations supporting patient active and passive data in according to Blocks 310 and 520 will be processed by the system 100, distributed across the method 200, analyzed throughout Block 501, and transmitted to Block 560 for Block 570.

The root cause analysis model, Block 525, is designed to generate predictive models in any suitable manner that define labels, values, relationships, and/or outputs, over a specified time period, to demonstrate a positive and/or negative connection between intervention and quality of life. Blocks 520 and 525 are designed as a feedback loop to be accessed by the system and method.

### 6.3 Adherence Analysis Module

As shown in FIG. 6C, the adherence analysis module, Block 530 is configured to receive and/or collect data from Block 310 and dataset 215. Additionally, Block 530 can automatically gather and/or collect data from 110, 120, 130, 213, 217, 231, 233, 410, and 415 to optimize Block 535, feedback analysis model.

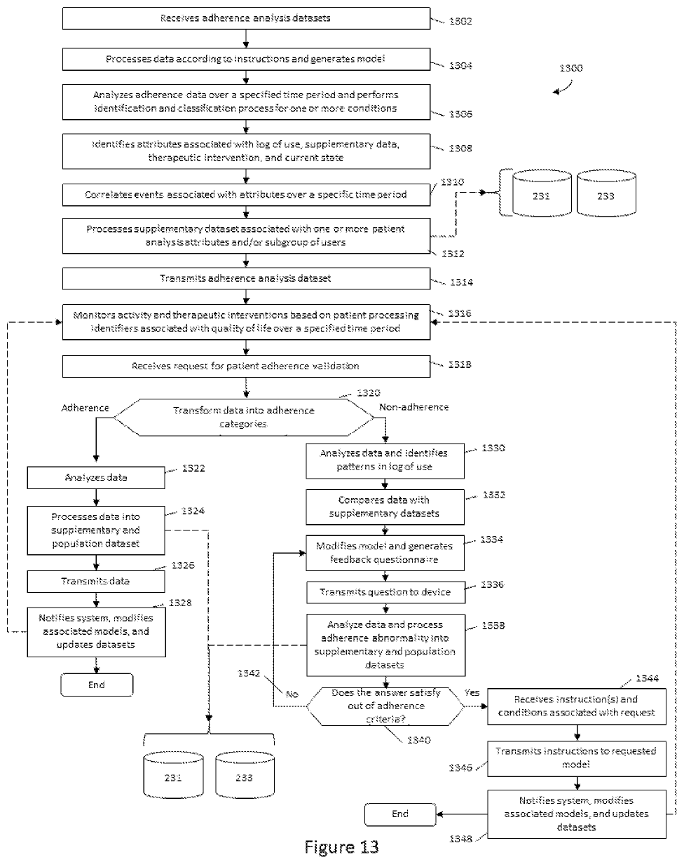

As shown in FIG. 13, an example procedure 1300 of the adherence method for Block 530 will include receives adherence analysis datasets 1302; processes data according to instructions and generates model 1304; analyzes adherence data over a specified time period and performs identification and classification process for one or more conditions 1306; identifies attributes associated with log of use, supplementary data, therapeutic intervention, and current state 1308; correlates events associated with attributes over a specific time period 1310; processes supplementary dataset associated with one or more patient analysis attributes and/or subgroup of users 1312; transmits adherence analysis dataset 1314; monitors activity and therapeutic interventions based on patient processing identifiers associated with quality of life over a specified time period 1316; receives request for patient adherence validation 1318; transforms data into adherence categories 1320, analyzes data based on positive adherence category 1322; processes data into supplementary and population dataset 1324; transmits data 1326; notifies system, modifies associated modules, and updates datasets 1328; analyzes data upon negative adherence category and identifies patterns in log of use 1330; compares data with supplementary datasets 1332; modifies model and generates feedback questionnaire 1334; transmits question to device 1336; analyzes data and process adherence abnormality into supplementary and population datasets 1338; does the answer satisfy out of adherence criteria? 1340; no 1342 and proceeds to 1334; yes answer notifies system, modifies associated models, and updates datasets 1344; receives instruction(s) and conditions associated with request 1346; and transmits instructions to requested model 1348.

Block 530 is configured to assess the improvement in patient quality of life over a specified time period according to therapeutic interventions. Block 530 is additionally configured to monitor patient responses to generate future therapeutic interventions based on patient active and passive datasets.

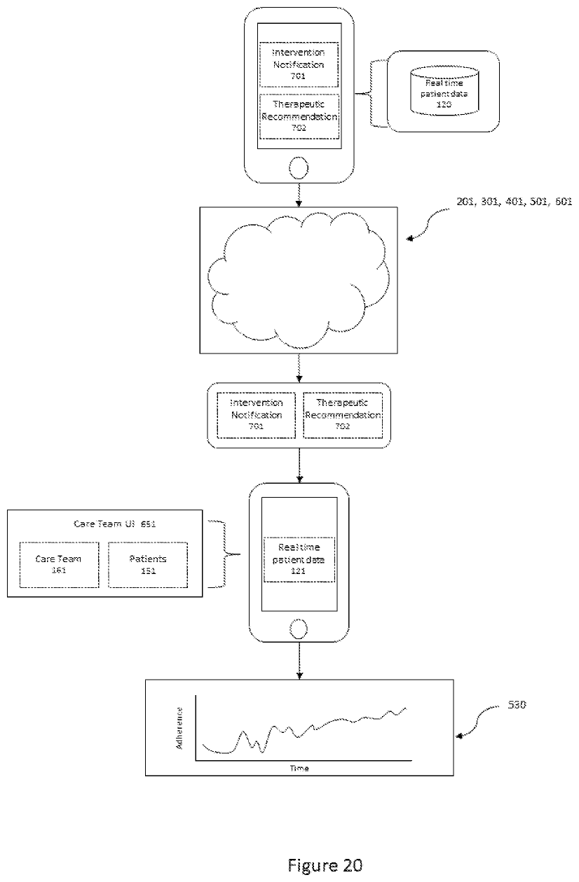

As shown in FIG. 20, regarding Block 530, generating an analysis is processed by one or more machine learning techniques, data mining, or statistical approaches to display the context of values, charts, and/or graphs highlighting the data.

Block 530 uses at least one of active data (e.g., health history dataset, symptom map dataset, migraine profile dataset, headache types dataset), passive data (e.g., digital application behaviors from log of use dataset; communication datasets from audio, text, and/or chatbot conversations; and supplementary dataset over a time period) to generate patient analysis information.

The adherence analysis model, Block 535, is designed to generate predictive models in any suitable manner that define labels, values, relationships, and/or outputs, over a specified time period, to demonstrate a positive and/or negative connection between activity, intervention, and quality of life. Blocks 530 and 535 are designed as a feedback loop to be accessed by the system and method.

### 6.4 Feedback Analysis Module

As shown in FIG. 6D, the patient feedback module, Block 540 is configured to receive and/or collect data from Block 410 and dataset 213. Additionally, Block 540 can automatically gather and/or collect data from 110, 120, 130, 213, 217, 231, and 233 to optimize Block 545, feedback analysis model.

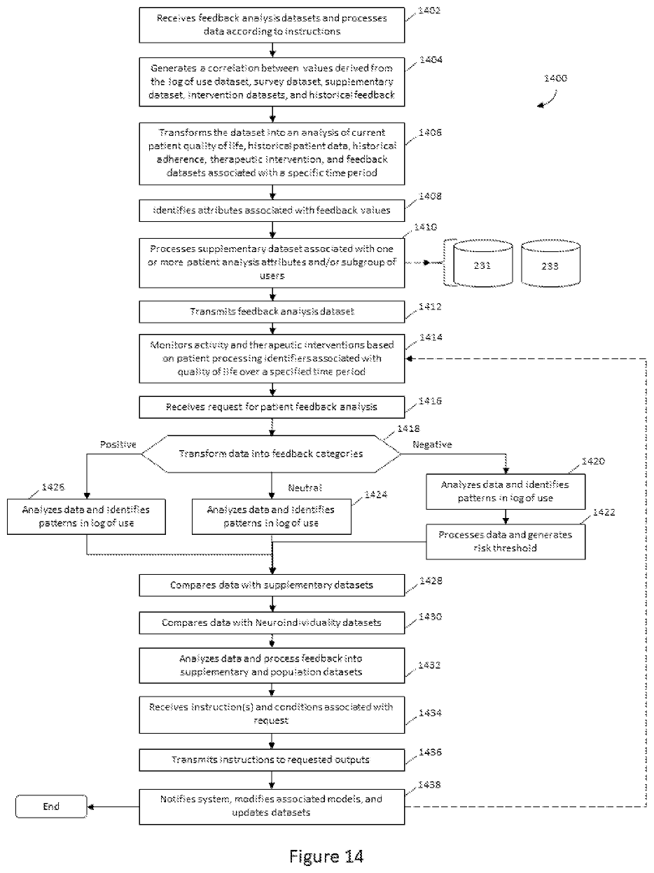

As shown in FIG. 14, an example procedure 1400 of the feedback method for Block 540 will include receives feedback analysis datasets and processes data according to instructions 1402; generates a correlation between values derived from the log of use dataset, survey dataset, supplementary dataset, intervention datasets, and historical feedback 1404; transforms the dataset into an analysis of current patient quality of life, historical patient data, historical adherence, therapeutic intervention, and feedback datasets associated with a specific time period 1406; identifies attributes associated with feedback values 1408; processes supplementary dataset associated with one or more patient analysis attributes and/or subgroup of users 1410; transmits feedback analysis dataset 1412; monitors activity and therapeutic interventions based on patient processing identifiers associated with quality of life over a specified time period 1414; receives request for patient feedback analysis 1416; transform data into feedback categories 1418; analyzes data and identifies patterns in log of use in a negative feedback category 1420; processes data and generates risk threshold 1422; analyzes data and identifies patterns in log of use in a neutral feedback category 1424; analyzes data and identifies patterns in log of use in a positive feedback category 1426; compares data with supplementary datasets 1428; compares data with Neuroindividuality datasets 1430; analyzes data and process feedback into supplementary and population datasets 1432; receive instruction(s) and conditions associated with request 1434; transmits instructions to requested outputs 1436; and notifies system, modifies associated models, and updates datasets 1438.

In relation to FIG. 2, Block 540 functions to receive feedback data, analyze, and transmit to Block 201. The system 100 will send modified instructions for Block 201 to process the data into appropriate feedback datasets 213. The feedback datasets can be used in any suitable manner by the system 100 and method 200 to update models to better improve patient quality of life and therapeutic lifestyle adherence.

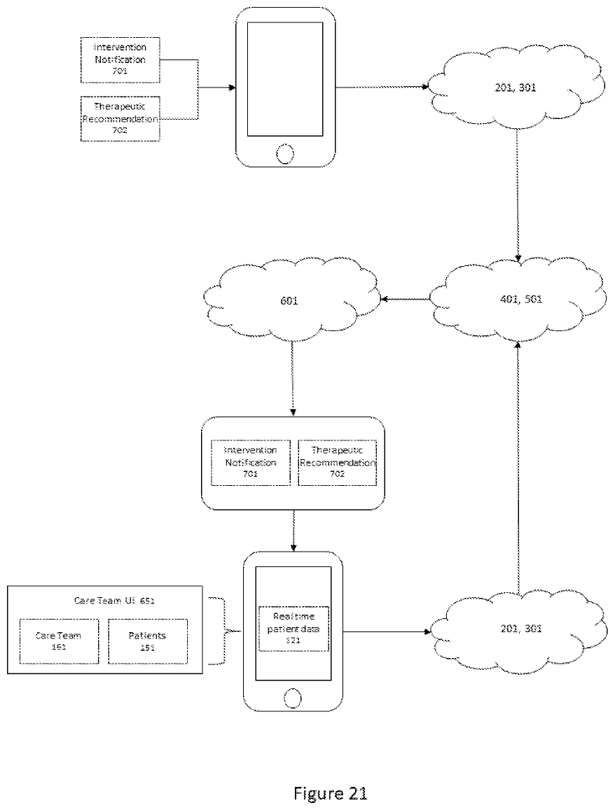

As shown in FIG. 21, the system view for processing feedback in a continuous loop transmits intervention notification 701 and therapeutic recommendation 702 to the patient digital computing device, data is transmitted to Blocks 201 and 301 for processing and analysis of patient data to determine feedback, log of use, improvement or decline in health status, change in migraine status, symptoms, and triggers. Once completed, the system 100 will transmit data to Blocks 401 and 501 to update Neuroindividuality and therapeutic intervention parameters in correlation and comparison with root cause and adherence. The system 100 will further process data with adherence, feedback and prediction analysis and deliver results to Block 560. If the system 100 determines the therapeutic intervention matches patient Neuroindividuality and intervention plan 570, data will be transmitted to Block 601 to determine optimal notification type, recommendation, and scheduling time. After all criteria is met, the system will transmit intervention notification and therapeutic recommendation. Real time patient interaction with mobile computing device will be processed, data will be stored in patient datasets 151, and the continuous feedback loop is initiated.

In one implementation, Block 540 will automatically generate a feedback prompt based on therapeutic lifestyle intervention. For example, Block 540 will generate a feedback prompt according to Blocks 220, 410, and 415 and monitor incoming feedback responses from dataset 120. The system will analyze the data for correlations in digital communication or content, feedback experience, and patient quality of life. Correlations with migraine and headache health metrics and feedback results, in accordance with patient's mental, spiritual, and/or physical states can include one or more of the migraine phases (prodrome, aura, pain, postdrome), migraine stigma status (e.g., mood, guilt, shame, emotions, symptoms associated with migraine-associated comorbid mental health, etc.), lifestyle habits (diet, exercise plans, self-care activity, breathwork, etc.), neurobiological status (e.g., lab tests, nutrition status, viruses, metabolic function, etc.), daily activity (planned activity, canceled activity, lack of activity, achieved activity, activity that improved or worsened migraine and headache pain levels, etc.). Block 501 will process the data from Blocks 510, 520, 530, 540, and 550 to generate Block 560 and modify Block 570.

In an example, patient with recurring sinus headaches, triggers (e.g., food sensitivity, and gluten), and primary symptom (sinus congestion), purchases new gluten-free bread and low-histamine snacks. Data received from 120 and processed by the system, identifies a potential connection between log of use, survey data, supplementary data, and Block 415 (e.g., purchase history, food ingredients, sinus headache, new snacks did not trigger sinus headache, increased happiness, and improved quality of life). Block 545 will receive instructions to generate feedback to see how the experience was with the purchase and if the patient should see more recommendations. The system will process and analyze feedback responses and transmit throughout the model.

The feedback analysis model, Block 545, is designed to generate predictive models in any suitable manner that define labels, values, relationships, and/or outputs, over a specified time period, to demonstrate a positive and/or negative connection between intervention and quality of life. Blocks 540 and 545 are designed as a feedback loop to be accessed by the system and method.

### 6.5 Prediction Analysis Module

As shown in FIG. 6E the prediction analysis module, Block 550 is configured to receive and/or collect patient survey, log of use, and supplementary datasets. Additionally, Block 550 can automatically gather and/or collect data from 110, 120, 130, 213, 231, 310, 320, 330, 415, 510, 520, 530, and 540 to optimize Block 555, prediction analysis model.

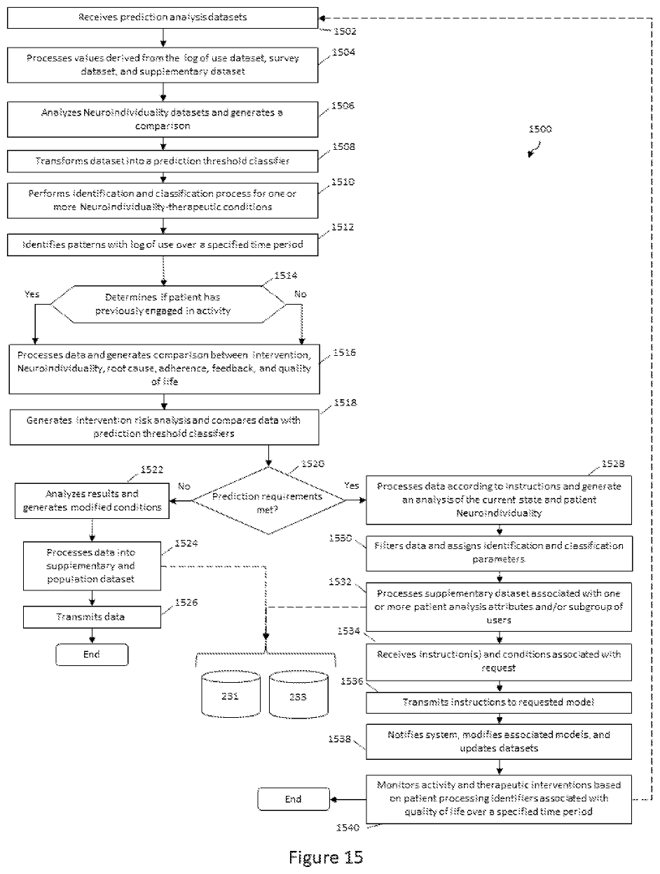

As shown in FIG. 15, an example procedure 1500 of the prediction method for Block 550 can include receives prediction analysis datasets 1502; processes values derived from the log of use dataset, survey dataset, and supplementary dataset 1504; analyzes Neuroindividuality datasets and generates a comparison 1506; transforms dataset into a prediction threshold classifier 1508; performs identification and classification process for one or more Neuroindividuality-therapeutic conditions 1510; identifies patterns with log of use over a specified time period 1512; determines if patient has previously engaged in activity 1514; processes data and generates comparison between intervention, Neuroindividuality, root cause, adherence, feedback, and quality of life 1516; generates intervention risk analysis and compares data with prediction threshold classifiers 1518; prediction requirements met? 1520; analyzes results and generate modified conditions 1522; processes data into supplementary and population dataset 1524; transmits data 1526; processes data according to instructions and generate an analysis of the current state and patient Neuroindividuality 1528; filters data and assigns identification and classification parameters 1530; processes supplementary dataset associated with one or more patient analysis attributes and/or subgroup of users 1532; receives instruction(s) and conditions associated with request 1534; transmits instructions to requested model 1536; notifies system, modifies associated models, and updates datasets 1538; monitors activity and therapeutic interventions based on patient processing identifiers associated with quality of life over a specified time period 1540

The prediction analysis model, Block 555, is designed to generate predictive models in any suitable manner that define labels, values, relationships, and/or outputs, over a specified time period, to demonstrate a positive and/or negative connection between intervention and quality of life. Blocks 550 and 555 are designed as a feedback loop to be accessed by the system and method.

### 6.6 Intervention Generation Module

As shown in FIGS. 6A-6E, the intervention generation module, Block 560, functions to identify migraine and headache type and migraine-associated comorbid therapeutic interventions with a patient.

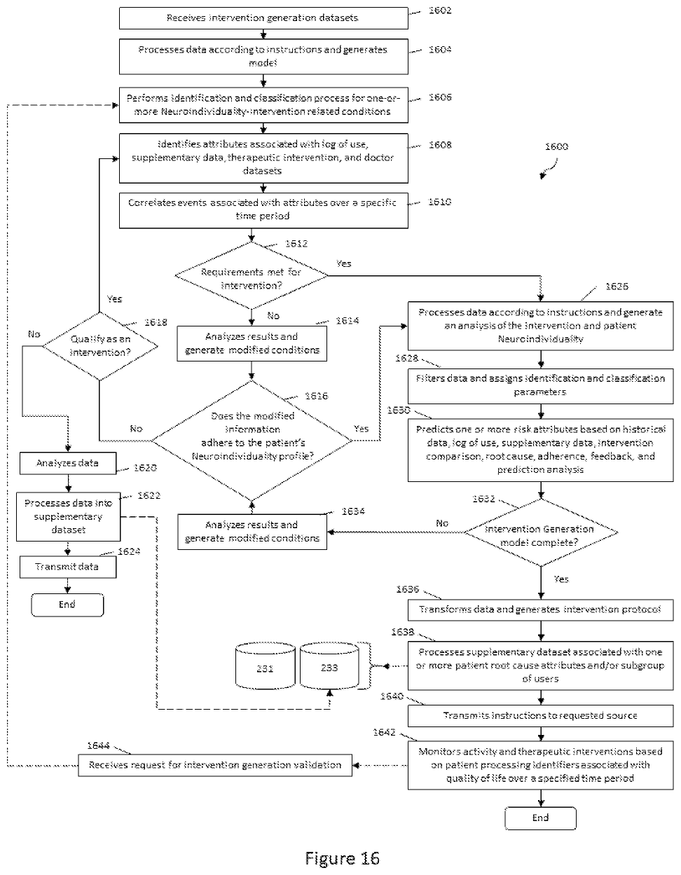

As shown in FIG. 16, an example procedure 1600 of the prediction method for Block 560 can include receives intervention generation datasets 1602; processes data according to instructions and generates model 1604; performs identification and classification process for one-or-more Neuroindividuality-intervention related conditions 1606; identifies attributes associated with log of use, supplementary data, therapeutic intervention, and doctor datasets 1608; correlates events associated with attributes over a specific time period 1610; requirements met for intervention? 1612; analyzes results and generate modified conditions 1614; does the modified information adhere to the patient's Neuroindividuality profile? 1616; qualify as an intervention? 1618; analyzes data 1620; processes data into supplementary dataset 1622; transmits data 1624; processes data according to instructions and generate an analysis of the intervention and patient Neuroindividuality 1626; filters data and assigns identification and classification parameters 1628; predicts one or more risk attributes based on historical data, log of use, supplementary data, intervention comparison, root cause, adherence, feedback, and prediction analysis 1630; intervention generation model complete? 1632; analyzes results and generate modified conditions 1634; transforms data and generates intervention protocol 1636; processes supplementary dataset associated with one or more patient root cause attributes and/or subgroup of users 1638; transmits instructions to requested source 1640; monitors activity and therapeutic interventions based on patient processing identifiers associated with quality of life over a specified time period 1642; receives request for intervention generation validation 1644.

For example, Block 560 can include generating a list of suitable migraine and headache therapeutic interventions from Block 410; filtering a list of potential therapeutics based on migraine and headache types, symptoms, lifestyle activity, current medication use, and/or any other suitable information; determining the likelihood of implementation based on socioeconomic information, demographic information, health insurance, past behavioral data, historic health history data, and/or any other suitable information; and updating Blocks 210, 220, 230, 320, 330, 415, 515, 525, 535, 545, and 555.

Additionally, Block 560 will determine the most applicable trigger factors associated with one or more therapeutic interventions. Once identified, one or more machine learning techniques, data mining, or statistical approaches are applied to develop a correlation. The labels, values, and/or weights will be used to process a series of percentages (e.g., readiness factor, risk, trigger, intervention success, etc.) associated with a patient's migraine and headache health status, migraine and headache triggers, and health profiles (e.g., health history, symptom map, headache type, etc.).

In a specific example, Block 560 can include identifying a patient's migraine and headache type (e.g., migraine with aura and tension-type headache) and migraine-associated comorbidity (e.g., anxiety, depression, and hypertension) to determine trends in stress response lifestyle activities as a trigger leading to future onset.

For example, Block 560 can include identifying a patient's migraine and headache type (e.g., migraine with aura, tension-type headache, menstrual migraine, etc.), frequency (e.g., chronic, etc.), dietary triggers (e.g., nitrates, high-histamine, high-sensitivity foods, etc.), lifestyle habit triggers (e.g., lack of self-care, poor stress management, excessive social media use, sedentary lifestyle, etc.), hormonal triggers (e.g., stress, low progesterone, etc.), sleep triggers (e.g., changes in sleep patterns, consuming coffee too late in the day, etc.), lab test data (e.g., low Vitamin B12, low Vitamin D, MTHFR gene mutation, etc.), preferred method of communication over a specific time period (e.g., push notification at 2:00 pm, etc.), and preferred type of learning content (e.g., audio with journaling exercise, etc.); determining the likelihood of intervention based on historical patient data and datasets from Block 201; employing a logical regression model; applying a weighted value to each potential intervention; categorizing the labels or values according to correlations found in patient data and/or patient population subsets; promoting the therapeutic intervention based on a log of use, communication datasets, and a patient's preferred communication and content method and type; modifying the intervention predictive model from patient survey datasets; and updating the intervention predictive model.

In a variation, Block 560, can include identifying potential subsets of the population, generating a predictive model from the previous log of use intervention dataset; applying labels or values based on patient survey responses; promoting an additional therapeutic intervention; generating an updated log of use intervention dataset to configure a therapeutic comparative model.

In another variation, Block 560, can be configured to generate one or more therapeutic intervention for migraine, headache, migraine-associated comorbidity, symptom, trigger, migraine lifestyle habit, and/or any other suitable intervention.

The system is configured to process data from Block 560 and transmit instructions to the intervention plan, Block 570.

### 6.7 Intervention Plan

As shown in FIGS. 6A-E and 7, the intervention plan, Block 570, functions to create an adaptive lifestyle therapeutic intervention plan, personalized to a patient's Neuroindividuality, providing physical, psychological, spiritual, and/or lifestyle medicine.

As shown in FIG. 6A-E and 7, Block 570 is configured to process and analyze one or more therapeutic interventions (e.g., selected in Blocks 401 and 560) to be administered through the patient's digital computing device and facilitated by care team 161. Block 570 will include at least one lifestyle therapeutic intervention (e.g., Block 410) according to Neuroindividuality (e.g., Block 310), trigger analysis (e.g., Block 320), headache type probability (e.g., Block 330), intervention comparison (e.g., Block 510), root cause (e.g., Block 520), adherence (e.g., Block 530), feedback (e.g., 540).

In some implementations, the distributed intervention plan would enable monitoring by the system to be facilitated by the care team 161. This will help train models and improve adherence and feedback to improve quality of life. In accordance to Block 570, as it relates to feedback, survey, and log of use datasets, the system 100 can process and escalate alerts to be generated and transmitted to the care team 161. The system 100 will process and analyze the data and results to modify thresholds and conditions for therapeutic intervention comparison 510, and Block 601.

In some implementations, population and supplementary datasets can provide modified actions of therapeutic intervention (e.g., Block 410 and 560). For example, the system analyzes population datasets and determines high probability of trends for at least one of a patient's Neuroindividuality, triggers, headache type, root cause, adherence, feedback survey, and/or log of use, modified instructions will be transmitted through the method 200. In a specific example, patient is non-responsive to current migraine medications and is reporting an increase in side-effect related symptoms. The system 100 will process and analyze population and supplementary datasets to determine if any potential correlations exist. If a potential correlation occurs, and is above a predetermined threshold, the system 100 can modify therapeutic intervention instructions to develop new parameters for therapeutic intervention generation. Additionally, the system 100 can generate alerts, notifications, and any other suitable content to be accessed by the care team 161 and the patient's mobile computing device.

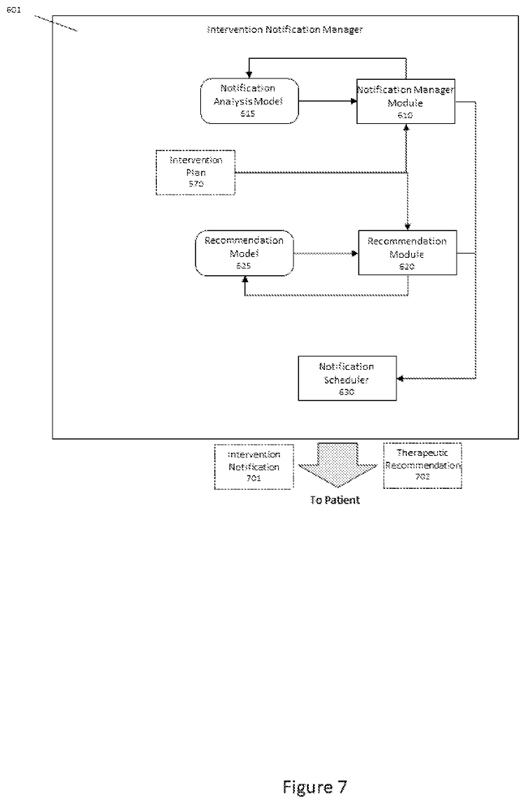

As shown in FIG. 7 Block 570 may send modified instructions according to notification manager module 610 and recommendation module 620. In an example, the system 100 can process data and generate an alternative logic in presenting the notification and/or recommendation to the patient (e.g., for adherence). Additionally, the system 100 can process alternative instructions for care team (e.g., tailor intervention to upcoming session) and family/friends/imported contacts/emergency contacts (e.g., status monitoring, accountability, etc.).

As an alternative configuration, Block 570 will be measured and monitored by the system 100 without the need for care team facilitation. For example, if the patient is practicing meditation, or another intervention deemed low risk, the system 100 can track and transmit updated results of their progress. After a predetermined threshold is met, the care team will be notified of progress.

### 7. Intervention Notification Manager

As shown in FIG. 7, the intervention notification manager, Block 601, consists of notification manager module 610, notification analysis model 615, recommendation module 620, recommendation model 625, and notification scheduler 630. Additionally Block 601 retrieves data from intervention plan 570 and processes intervention notification 701 and therapeutic recommendation 702 to the patient.

### 7.1 Notification Manager Module

As shown in FIG. 7, the notification manager module, Block 610, functions to identify the most optimal notification type to increase adherence and improvement in quality of life.

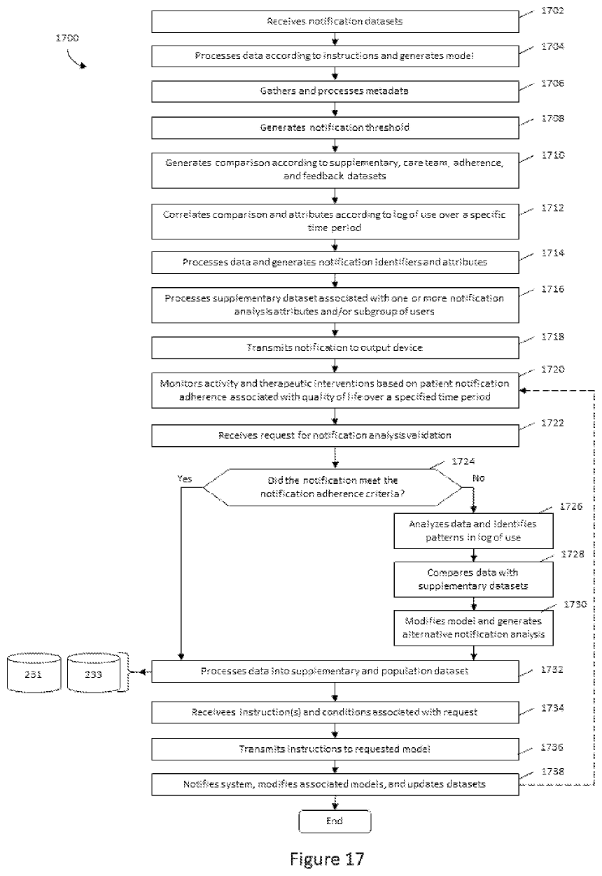

As shown in FIG. 17 an example procedure 1700 of the prediction method for Block 610 can include receives notification datasets 1702; processes data according to instructions and generates model 1704; gathers and processes metadata 1706; generates notification threshold 1708; generates comparison according to supplementary, care team, adherence, and feedback datasets 1710; correlates comparison and attributes according to log of use over a specific time period 1712; processes data and generates notification identifiers and attributes 1714; processes supplementary dataset associated with one or more notification analysis attributes and/or subgroup of users 1716; transmits notification to output device 1718; monitors activity and therapeutic interventions based on patient notification adherence associated with quality of life over a specified time period 1720; receives request for notification analysis validation 1722; did the notification meet the notification adherence criteria? 1724; analyzes data and Identifies patterns in log of use 1726; compares data with supplementary datasets 1728; modifies model and generates alternative notification analysis 1730; processes data into supplementary and population dataset 1732; receives instruction(s) and conditions associated with request 1734; transmits instructions to requested model 1736; notifies system, modifies associated models, and updates datasets 1738.

In an example, Block 610 will select from a notification type (e.g., in-app alert, text message, email, phone call, video call, push notification, message, chatbot prompt, care team member message, activity prompt, marketplace discount alert, upcoming appointment reminder, breaking news, population alert, etc.) according to feedback, adherence, log of use, and supplementary datasets.

In a variation, Block 610 will process patient feedback (e.g., wants more text messages), most adheres to (e.g., in-app alert, push notification, and text message), with increased log of use (e.g., increased use of chatbot) and generate a set of identifiers to be used by Blocks 620 and 630.

The notification manager model, Block 615, is designed to generate predictive models in any suitable manner that define labels, values, relationships, and/or outputs, over a specified time period, to demonstrate a positive and/or negative connection between intervention and quality of life. Blocks 610 and 615 are designed as a feedback loop to be accessed by the system 100 and method 200.

### 7.2 Recommendation Module

As shown in FIG. 7, the recommendation module, Block 620, is configured to generate recommendation identifiers and classifications according to adherence, feedback, log of use, care team, and supplementary datasets to determine the optimal recommendation type for the patient.

The term recommendation type can include any one or more of the following content type (e.g., infographic, video, audio, challenge, exercise, badge), treatment (e.g., abortive and/or preventative medications), therapeutic intervention (e.g., chiropractic, journaling, breathwork, exercise, stretching, etc.) and/or lifestyle habits (e.g., food, supplements, physical activity, etc.) and/or any other suitable recommendation type determined by the system and/or care team.

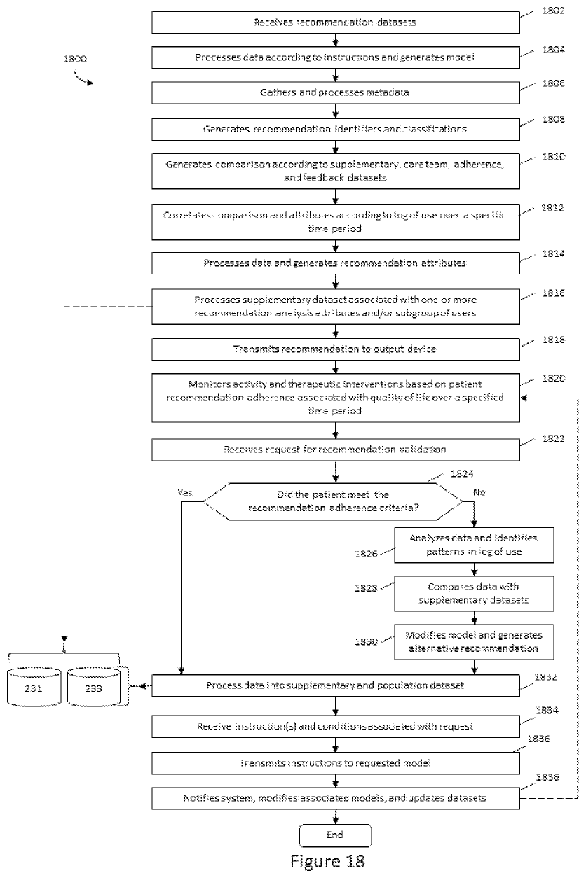

As shown in FIG. 18 an example procedure 1800 of the prediction method for Block 620 can include receives recommendation datasets 1802; processes data according to instructions and generates model 1804; gathers and processes metadata 1806; generates recommendation identifiers and classifications 1808; generates comparison according to supplementary, care team, adherence, and feedback datasets 1810; correlates comparison and attributes according to log of use over a specific time period 1812; processes data and generates recommendation attributes 1814; processes supplementary dataset associated with one or more recommendation analysis attributes and/or subgroup of users 1816; transmits recommendation to output device 1818; monitors activity and therapeutic interventions based on patient recommendation adherence associated with quality of life over a specified time period 1820; receives request for recommendation validation 1822; did the patient meet the recommendation adherence criteria? 1824; analyzes data and Identifies patterns in log of use 1826; compares data with supplementary datasets 1828; modifies model and generates alternative recommendation 1830; processes data into supplementary and population dataset 1832; receives instruction(s) and conditions associated with request 1834; transmits instructions to requested model 1836; notifies system, modifies associated models, and updates datasets 1838.

In a specific example, patient has reported an increase in tension-type headaches during work. According to patient real time data and feedback, adherence, log of use, and supplementary datasets, the system 100 has identified high probability of triggers (e.g., poor posture, too much sitting, blood sugar fluctuations, stressful meetings, too much screen time) with potential root causes (e.g., lack of meal preparation to avoid blood sugar fluctuations, lack of awareness to posture at desk, not enough breaks in between long periods of sitting and computer use, lack of mindfulness prior to stressful meeting). The system 100 has further identified patient is currently working with the care team 161 (e.g., dietician for meal planning, health coach for habit modification, and mental health professional for mindfulness implementation). After further processing, the system 100 has identified a breathwork exercise prior to the upcoming meeting as a high probability recommendation for adherence and positive feedback, while in accordance with the patient's Neuroindividuality 310 and treatment plan 570.

The recommendation manager model, Block 625, is designed to generate predictive models in any suitable manner that define labels, values, relationships, and/or outputs, over a specified time period, to demonstrate a positive and/or negative connection between patient application activity, intervention, and quality of life. Blocks 620 and 625 are designed as a feedback loop to be accessed by the system 100 and method 200.

### 7.3 Notificaiton Scheduler

As shown in FIG. 7, the notification scheduler, Block 630, is configured to transmit notification(s) to the patient's mobile computing device at optimal time period to increase adherence and quality of life according to information processed by Block 610 and 620. Block 630 functions to correlate patient digital application log of use with one or more of supplementary, adherence, and/or feedback datasets. The system 100 will process instructions to Block 601 in preparation to deliver intervention notification and therapeutic recommendation to the patient.

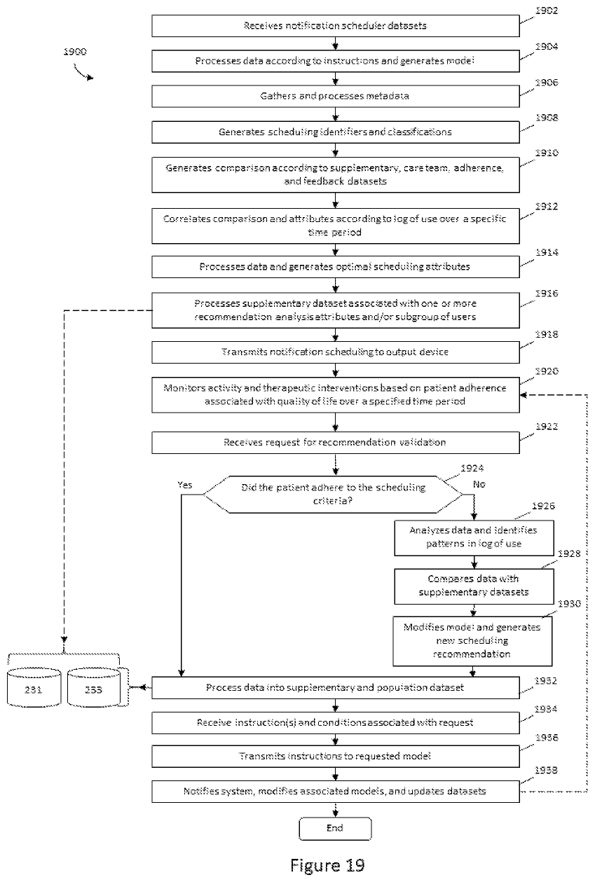

As shown in FIG. 19 an example procedure 1900 of the prediction method for Block 620 can include receives notification scheduler datasets 1902; processes data according to instructions and generates model 1904; gathers and processes metadata 1906; generates scheduling identifiers and classifications 1908; generates comparison according to supplementary, care team, adherence, and feedback datasets 1910; correlates comparison and attributes according to log of use over a specific time period 1912; processes data and generates optimal scheduling attributes 1914; processes supplementary dataset associated with one or more recommendation analysis attributes and/or subgroup of users 1916; transmits notification scheduling to output device 1918; monitors activity and therapeutic interventions based on patient adherence associated with quality of life over a specified time period 1920; receives request for recommendation validation 1922; did the patient adhere to the scheduling criteria? 1924; analyzes data and Identifies patterns in log of use 1926; compares data with supplementary datasets 1928; modifies model and generates new scheduling recommendation 1930; processes data into supplementary and population dataset 1932; receives instruction(s) and conditions associated with request 1934; transmits instructions to requested model 1936; notifies system, modifies associated models, and updates datasets 1938.

Block 630 is configured to automatically modify scheduling instructions in accordance with recommendation, notification type, and intervention. For example, according to log of use datasets 217, patient has been most active during the hours of 7 pm and 8:30 pm and has best responded to in-app alerts. Based on those parameters, the system 100 will create classifications and identifiers to rank therapeutic interventions and deliver an optimal experience to improve adherence. If the therapeutic intervention is a breathwork activity prior to a work meeting at 1 pm, the system will determine if the patient is using the app, based on high adherence of response to in-app notifications. Upon determination, the patient is not currently using the app when the breathwork intervention is scheduled to be delivered, generating a modified notification type to be processed. The system 100 will process modified instructions and deliver therapeutic intervention to patient in Blocks 701 and 702.

### 7.4 Intervention Notification

As shown in FIGS. 2 and 7, intervention notification, Block 701, functions to retrieve, analyze, and transmit intervention notification to the patient. Block 701 is configured by the system 100 and method 200 to transmit selected notification to be accessed by the patient through the mobile computing device 30 and/or any other remote devices 40 where the application is installed.

As shown in FIG. 19, in relation to the intervention notification, Block 701, once the patient interacts with the application on the mobile computing device 30, real time patient data 120 will be collected and processed by the system 100, method 200, and Blocks 201, 301, 401, 501, 601 to deliver continuous and automatic intervention notifications 701 and therapeutic recommendations 702. Once patient interacts with the application on the mobile computing device 30, real time data is collected 121, transmitted to care team UI 651, and analyzed by Block 530 for adherence. Positive adherence for therapeutic recommendation will result in further training of models and personalization.

Additionally, Block 701 will store all patient notification data to be accessed by the system 100 and analyzed in comparison to feedback, adherence, survey, and log of use datasets. In some implementations, the system 100 will process the data to modify notification models according to population and supplementary datasets. The system 100 will process and transmit data throughout the method to improve adherence and patient quality of life.

Additionally, Block 701 can interact with Block 702 to gather historic and real time patient data as it relates to log of use to further optimize models.

In relation to Block 701, the system can generate one or more notification evaluations to be performed automatically and continuously, in response to care team (e.g., updating patient data), log of use (e.g., change in user behavior), adherence (e.g., decreased adherence for a once popular intervention), and/or any other suitable notification evaluation criteria.

### 7.5 Therapeutic Recommendation

As shown in FIGS. 2 and 7, therapeutic recommendation, Block 702, functions to retrieve, analyze, and transmit intervention recommendation to the patient. Block 702 is configured by the system 100 and method 200 to transmit selected recommendation to be accessed by the patient through the mobile computing device 30 and/or any other remote devices 40 where the application is installed.

As shown in FIG. 19, in relation to the therapeutic recommendation, Block 702, once the patient interacts with the application on the mobile computing device 30, real time patient data 120 will be collected and processed by the system 100, method 200, and Blocks 201, 301, 401, 501, 601 to deliver continuous and automatic intervention notifications 701 and therapeutic recommendations 702. Once patient interacts with the application on the mobile computing device 30, real time data is collected 121, transmitted to care team UI 651, and analyzed by Block 530 for adherence. Positive adherence for therapeutic recommendation will result in further training of models and personalization.

Additionally, Block 702 will store all patient recommendation data to be accessed by the system 100 and analyzed in comparison to feedback, adherence, survey, and log of use datasets. In some implementations, the system 100 will process the data to modify recommendation models according to population and supplementary datasets. The system 100 will process and transmit data throughout the method 200 to improve adherence and patient quality of life.

Additionally, Block 702 can interact with Block 701 to gather historic and real time patient data as it relates to log of use to further optimize models.

In relation to Block 702, the system can generate one or more recommendation evaluations to be performed automatically and continuously, in response to care team (e.g., updating patient data), log of use (e.g., change in user behavior), adherence (e.g., decreased adherence for a once popular intervention), and/or any other suitable recommendation evaluation criteria.

### 8. Care Team UI

As shown in FIG. 2, the care team UI, Block 651, consists of care team 161 and patients 151, and functions to act as the electronic patient monitoring system for digital migraine headache therapeutic lifestyle management. Block 651 is configured to access, retrieve, deploy, communicate, analyze, and transmit patient data processed by the system 100 and method 200. The system and architecture for Block 651 is developed for privacy, security, and adherence with all HIPAA compliance and telehealth regulations. Additionally, Block 651 personal health records and patient health conditions can be retrieved through the Fast Healthcare Interoperability Resources (FHIR) API.

Block 651 additionally functions for care team members to maintain patient health and medical records; collaborative interaction with care team members 161; facilitate telehealth sessions; prescription medication monitoring and processing; and integration with patient mobile computing device to deliver therapeutic interventions, content, alerts, notifications, messages, and any other suitable interface interaction.

### 8.1 Care Team

As shown in FIG. 2, the care team, Block 161, consists of the following care team members: clinician (medical doctor, neurologist, headache specialist, nurse practitioner, nurse), mental health professional (psychiatrist, psychologist, licensed clinical social worker, and/or any other mental health professional), dietician, health coach, and care coordinator.

The system and architecture of Block 651, as it relates to the care team members 151, is determined based on credentials and user permissions. Additionally, the care team members will have limited access to patient information and functionality in delivering, facilitating, and/or recommending interventions. For example, a health coach and dietician will have no access to modify and/or prescribe medication(s). The clinician will have no access to mental health patient session notes.

In a variation, the patient can have the authority to give consent for further collaboration and record sharing amongst care team members, but all interactions will follow healthcare laws and regulations.

Additionally, Block 651 is designed to optimize remote monitoring and allow for the collaboration of care team members 161 to treat symptoms with allopathic treatments, lifestyle medicine, and digital therapeutic interventions.

Additionally, Block 151 will store all care team interaction data (voice, text, messages, recommendations, etc.) to be used by the system 100 and analyzed to further train and develop models.

### 8.2 Patients

As shown in FIG. 2, patients, Block 151, functions to deliver digital application data to the care team UI 651 and accessed by care team 161. Block 151 is designed to aggregate many types of patient data (e.g., historical, real time, adherence, survey, log of use, etc.) and transmit according to the architecture of Block 651. Block 651 is optimized to act as a central storage, retrieval, and processing location for patient health information to monitor, deliver, and facilitate care to improve patient quality of life.

Additionally, Block 151 is configured to access, process, and analyze datasets (e.g., historical, real time, log of use, population, supplementary) according to patient-specific rules and transmit results to Block 651 to optimize care delivery for care team 161.

Block 651 will allow for the joint interaction between care team 151 and patients 161. For example, all information that is completed, reviewed, received, in-review, in-process, awaiting approval, and/or any other status identifier will be visible within the care team UI and the patient's mobile computing device.

1. A digital therapeutic computer system for treating migraine in a patient comprising:
a migraine headache data collector configured to receive historical patient data, real time patient data, and external data, wherein the historical patient data and the real time patient data are received through a digital therapeutic mobile application on a mobile device associated with the patient and wherein the external data is received from a source external to the mobile device and the digital therapeutic system;
a migraine headache analyzer comprising a neuroindividuality module configured to generate a neuroindividuality model, a trigger analysis module configured to generate a trigger model, and a headache type probability module configured to generate a headache type probability model, wherein one or more of the neuroindividuality model, the trigger model, and the headache type probability model are generated based on one or more of the historical patient data, the real time patient data, and the external data;
a therapeutic intervention module, in communication with the migraine headache analyzer, configured to determine a therapeutic intervention indicator and comprising multiple machine-learning modules; and
a notification module configured to transmit the therapeutic intervention indicator to the digital therapeutic mobile application on the mobile device associated with the patient or to a user interface on a computer system associated with a medical care team for the patient.

2. The digital therapeutic system of claim 1, wherein the historical patient data is associated with a first time period, the real time patient data is associated with a second time period, and the external data is associated with a third time period, and wherein one or more of the neuroindividuality model, the trigger model, and the headache type probability model are generated based on the first, second, and third time periods.

3. The digital therapeutic system of claim 1, wherein the migraine headache analyzer further comprises generating a migraine health metric by applying a regression, clustering, or learning function to the historical patient data, the real time patient data, and the external data to determine migraine or headache type, or migraine-associated co-morbidity.

4. The digital therapeutic system of claim 1, wherein the therapeutic intervention module further comprises multiple domain expert modules configured to apply expert rule to the historical and real time patient data and to the external data, to select the therapeutic intervention indicator, and to determine whether to communicate the intervention indicator to the mobile application on the mobile device associated with the patient or to the user interface on the computer system associated with the medical care team for the patient, or to both the mobile application and the user interface.

5. The digital therapeutic system of claim 1, wherein the notification module further comprises a scheduling module configured to schedule the transmission of notifications based on frequency of user activity within the mobile application and first and second methods of engagement with the mobile application.

6. A digital therapeutic computer method for treating migraine in a patient comprising:
receiving historical patient data, real time patient data, and external data, wherein the historical patient data and the real time patient data are received through a digital therapeutic mobile application on a mobile device associated with the patient and wherein the external data is received from a source external to the mobile device;
generating a neuroindividuality model, a trigger model, and a headache type probability model, wherein one or more of the neuroindividuality model, the trigger model, and the headache type probability model are generated based on one or more of the historical patient data, the real time patient data, and the external data;
determining, in communication with the neuroindividuality model, the trigger model, and the headache type probability model using multiple machine-learning modules; and
transmitting the therapeutic intervention indicator to the digital therapeutic mobile application on the mobile device associated with the patient or to a user interface on a computer system associated with a medical care team for the patient.

7. The digital therapeutic computer method of claim 6, wherein the historical patient data is associated with a first time period, the real time patient data is associated with a second time period, and the external data is associated with a third time period, and wherein one or more of the neuroindividuality model, the trigger model, and the headache type probability model are generated based on the first, second, and third time periods.

8. The digital therapeutic computer method of claim 6, further comprising generating a migraine health metric by applying a regression, clustering, or learning function to the historical patient data, the real time patient data, and the external data to determine migraine or headache type, or migraine-associated co-morbidity.

9. The digital therapeutic computer method of claim 6, applying expert rule to the historical and real time patient data and to the external data, selecting the therapeutic intervention indicator, and determining whether to communicate the intervention indicator to the mobile application on the mobile device associated with the patient or to the user interface on the computer system associated with the medical care team for the patient, or to both the mobile application and the user interface.

10. The digital therapeutic computer method of claim 6, further comprising scheduling the transmission of notifications based on frequency of user activity within the mobile application and first and second methods of engagement with the mobile application.

11. One or more computer-readable media having stored thereon executable instructions that when executed by one or more processors configure a computer system to perform at least the following:
receive historical patient data, real time patient data, and external data, wherein the historical patient data and the real time patient data are received through a digital therapeutic mobile application on a mobile device associated with the patient and wherein the external data is received from a source external to the mobile device;
generate a neuroindividuality model, a trigger model, and a headache type probability model, wherein one or more of the neuroindividuality model, the trigger model, and the headache type probability model are generated based on one or more of the historical patient data, the real time patient data, and the external data;
determine, in communication with the neuroindividuality model, the trigger model, and the headache type probability model using multiple machine-learning modules; and
transmit the therapeutic intervention indicator to the digital therapeutic mobile application on the mobile device associated with the patient or to a user interface on a computer system associated with a medical care team for the patient.

12. The computer-readable media of claim 11, wherein the historical patient data is associated with a first time period, the real time patient data is associated with a second time period, and the external data is associated with a third time period, and wherein one or more of the neuroindividuality model, the trigger model, and the headache type probability model are generated based on the first, second, and third time periods.

13. The computer-readable media of claim 11, further having stored thereon executable instructions that when executed by the one or more processors configure the computer system to perform at least the following:
generate a migraine health metric by applying a regression, clustering, or learning function to the historical patient data, the real time patient data, and the external data to determine migraine or headache type, or migraine-associated co-morbidity.

14. The computer-readable media of claim 11, further having stored thereon executable instructions that when executed by the one or more processors configure the computer system to perform at least the following:
apply expert rule to the historical and real time patient data and to the external data;
select the therapeutic intervention indicator; and
determine whether to communicate the intervention indicator to the mobile application on the mobile device associated with the patient or to the user interface on the computer system associated with the medical care team for the patient, or to both the mobile application and the user interface.

15. The computer-readable media of claim 11, further having stored thereon executable instructions that when executed by the one or more processors configure the computer system to perform at least the following:
schedule the transmission of notifications based on frequency of user activity within the mobile application and first and second methods of engagement with the mobile application.
